(19) United States
(12) Patent Application Publication
(10) Pub. No.: US 2017/0003589 A1
(43) Pub. Date: Jan. 5, 2017

(54) PHOTOINITIATOR AND PHOTOSENSITIVE COMPOSITION INCLUDING THE SAME

(71) Applicant: TAKOMA TECHNOLOGY CO., LTD., Nonsan (KR)

(72) Inventors: Mi Sun YOO, Daejeon (KR); Bok Joo SONG, Daejeon (KR)

(21) Appl. No.: 15/100,305

(22) PCT Filed: Nov. 27, 2014
(86) PCT No.: PCT/KR2014/011511

§ 371 Date: May 28, 2016

(30) Foreign Application Priority Data
Nov. 28, 2013 (KR) 10-2013-0146567
Nov. 5, 2014 (KR) 10-2014-0153212

(51) Int. Cl. G03F 7/031; C07D 409/06; C07D 209/86

(52) U.S. Cl. CPC G03F 7/031; C07D 209/86; C07D 409/06

(57) ABSTRACT

Provided is a photoinitiator represented by Formula 1:

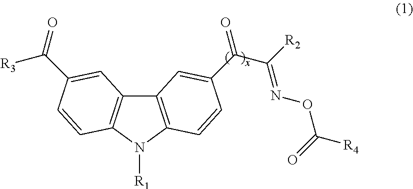

The photoinitiator is suitable for photocrosslinking. Further provided is a photosensitive resin composition including the photoinitiator. The photoinitiator and the photosensitive resin composition have improved solubility and high sensitivity. The photosensitive resin composition is suitable for use in the production of black resists, color resists, overcoats, column spacers, and organic insulating films for LCDs.

### BACKGROUND OF THE INVENTION

1. Field of the Invention

The present invention relates to a photoinitiator and a photosensitive composition including the same.

2. Description of the Related Art

Photosensitive compositions are prepared by adding a photoinitiator to a polymerizable compound having an ethylenically unsaturated bond. Such photosensitive compositions can be polymerized and cured upon irradiation with polychromatic light with wavelengths of 365 nm, 405 nm, and 436 nm and are thus used in photocurable inks, photosensitive printing plates, various types of photoresists, etc. Photosensitive compositions sensitive to short-wavelength light sources can be micro-printed. Thus, photopolymerization initiators are particularly needed that have high sensitivity to short-wavelength light sources, particularly, 365 nm light sources. Many oxime ester compounds are used as highly sensitive photoinitiators. Numerous characteristics of oxime ester compounds are described in several patent publications and some commercial products for oxime ester compounds are known.

Most of such oxime ester photoinitiators are currently applied to photoresists in the field of LCDs. Commercially available products for oxime ester photoinitiators are divided into α-ketoxime ester compounds and oxime ester compounds. α-ketoxime ester photoinitiators are used in color photoresists, mainly red, green, and blue photoresists.

Oxime ester compounds may be decomposed when irradiated with UV light. This photodecomposition changes the color of resist films. In contrast, α-ketoxime ester photoinitiators do not tend to discolor when exposed to UV light, causing no change in the color coordinates of color resists. For this reason, α-ketoxime ester compounds are mainly used in color photoresists. However, currently commercially available α-ketoxime ester photopolymerization initiators suffer from the problem of low sensitivity. Under these circumstances, a need exists for highly sensitive α-ketoxime ester photopolymerization initiators.

### SUMMARY OF THE INVENTION

Photoresist compounds undergo photocuring to form patterns when irradiated with UV. Highly sensitive photoinitiators with high photoreactivity and easy-to-prepare and easy-to-handle photoinitiators with high solubility are required to shorten the processing time of photocuring. For example, when photoresist compounds are applied to color resists, resists are needed which include pigments dispersed by advanced techniques in order to achieve high color quality characteristics. A higher pigment content tends to make the curing of a color resist more difficult. Thus, there is a need for initiators that have higher photosensitivity than general-use initiators. Such photoinitiators are required to meet stringent requirements in terms of industrially relevant characteristics, such as high solubility in organic solvents and good thermal and storage stability.

It is an object of the present invention to provide an α-ketoxime ester compound or an oxime ester compound as a highly sensitive photoinitiator that has a UV absorbance peak at a wavelength close to 365 nm to 410 nm while possessing excellent characteristics in terms of developability, adhesiveness, and alkali resistance.

According to one aspect of the present invention, there is provided a photoinitiator represented by Formula 1:

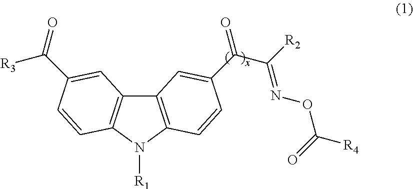

wherein R₁ is a C₁-C₁₂ linear, branched or cyclic alkyl group that may contain oxygen, sulfur, nitrogen or an ester bond in the chain, R₂ is a C₁-C₆ alkyl group; a C₆-C₂₀ aryl group that may be optionally substituted with oxygen, sulfur, nitrogen, a C₁-C₃ alkyl group, a nitro group or a halogen atom; a 2-methylbenzyl group;

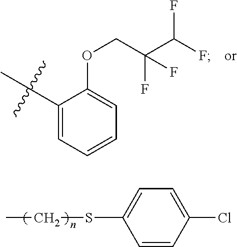

(n=1-4), R₃ is a C₁-C₁₀ linear, branched or cyclic alkyl group, a C₆-C₂₀ aryl group, or a C₄-C₂₀ heteroaryl group, R₄ is a C₁-C₁₀ linear, branched or cyclic alkyl group or a phenyl group, and x is 0 or 1.

According to another aspect of the present invention, there is provided a photosensitive resin composition including the photoinitiator represented by Formula 1.

The oxime ester-based photoinitiator of the present invention is highly soluble in solvents (e.g., PGMEA) suitable for use in photosensitive compositions. As a result, the required amount of the oxime ester-based photoinitiator for photocros slinking can be minimized When the photosensitive composition of the present invention is coated and evaporated to remove a solvent, the occurrence of phase separation between a binder and the photoinitiator can be reduced, resulting in improved thin film characteristics. The use of the photosensitive composition enables the production of high quality black matrices, color filters, column spacers, insulating films, photocrosslinkable films, etc.

### DETAILED DESCRIPTION OF THE INVENTION

The present invention provides an α-ketoxime ester compound or an oxime ester compound as an photoinitiator that can simultaneously meet requirements in terms of solubility in organic solvents and photosensitivity.

The present invention also provides a photosensitive resin composition including the photoinitiator and a photopolymerizable compound having an ethylenically unsaturated bond.

Specifically, the photoinitiator of the present invention is represented by Formula 1:

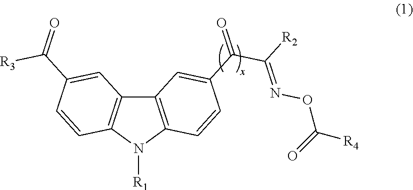

The photoinitiator represented by Formula 1 includes an α-ketoxime ester structure or an oxime ester structure.

In Formula 1, x is 0 or 1. When x is 0, the photoinitiator of Formula 1 is an oxime ester compound. When x is 1, the photoinitiator of Formula 1 is an α-ketoxime ester compound.

R₁ is a C₁-C₁₂ linear, branched or cyclic alkyl group that may contain oxygen, sulfur, nitrogen or an ester bond in the chain. In preferred embodiments, x may be 1 and R₁ may be a C₁-C₆ alkoxyalkyl or acyloxyalkyl group.

R₂ is a C₁-C₆ alkyl group; a C₆-C₂₀ aryl group that may be optionally substituted with oxygen, sulfur, nitrogen, a C₁-C₃ alkyl group, a nitro group or a halogen atom; a 2-methylbenzyl group;

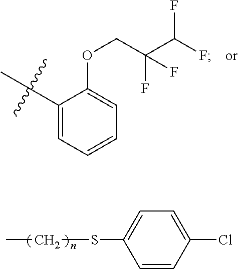

(n=1-4).

R₃ is a C₁-C₁₀ linear, branched or cyclic alkyl group, a C₆-C₂₀ aryl group, or a C₄-C₂₀ heteroaryl group. Preferably, R₃ is a thienyl, naphthyl, tolyl, or C₆-C₂₀ aryl group in which the aryl group is optionally substituted with a fluoro group, a fluorinated alkyl group, or a fluorinated alkoxy group.

R₄ is a C₁-C₁₀ linear, branched or cyclic alkyl group or a phenyl group.

The term “aryl” means, unless otherwise stated, a polyunsaturated, aromatic, hydrocarbon substituent which can be a single ring or multiple rings (from 1 to 3 rings) which are fused together or linked covalently. The term “heteroaryl” refers to aryl groups (or rings) that contain from one to four heteroatoms selected from N, O, and S, werein the nitrogen and sulfur atoms are optionally oxidized, and the nitrogen atom(s) are optionally quaternized. A heteroaryl group can be attached to the remainder of the molecule through carbon or a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Unless otherwise mentioned, the alkyl, aryl, heteroaryl, and alkoxyalkyl groups are intended to include both substituted and unsubstituted ones.

The term “substituted” means that one or more hydrogen atoms in the hydrocarbon are each independently replaced by the same or different substituents. Suitable substituents include, but are not limited to, fluoro, chloro, bromo, cyano, nitro, hydroxyl, amino, alkoxy, halogenated alkyl, and halogenated alkoxy groups.

R₂ in Formula 1 may be an aryl group, and specific examples thereof include phenyl, p-methoxyphenyl, p-fluorophenyl, p-bromophenyl, pentafluorophenyl, biphenyl, 1-naphthyl, 2-naphthyl, 9-anthryl, 9-phenanthryl, 1-pyrenyl, 5-naphthacenyl, 1-indenyl, 2-azulenyl, 9-fluorenyl, terphenyl, o-tolyl, m-tolyl, p-tolyl, xenyl, o-cumenyl, m-cumenyl, p-cumenyl, mesityl, pentalenyl, binaphthalenyl, ternaphthalenyl, biphenylenyl, indacenyl, fluoranthenyl, acenaphthylenyl, fluorenyl, anthryl, bianthracenyl, teranthracenyl, anthraquinolyl, phenanthryl, triphenylenyl, p-phenylthiophenyl, 2-(2,2,3,3-tetrafluoropropoxy)benzoyl, 2,3,4,5,6-pentapentafluorobenzoyl, o-(trifluoromethyl)benzoyl, m-(trifluoromethyl)benzoyl, and p-(trifluoromethyl)benzoyl groups.

R₃ in Formula 1 may represent an aryl or heteroaryl group. Specifically, R₃ can be selected from the following structural formulae:

(Exemplary Structures of R₃)

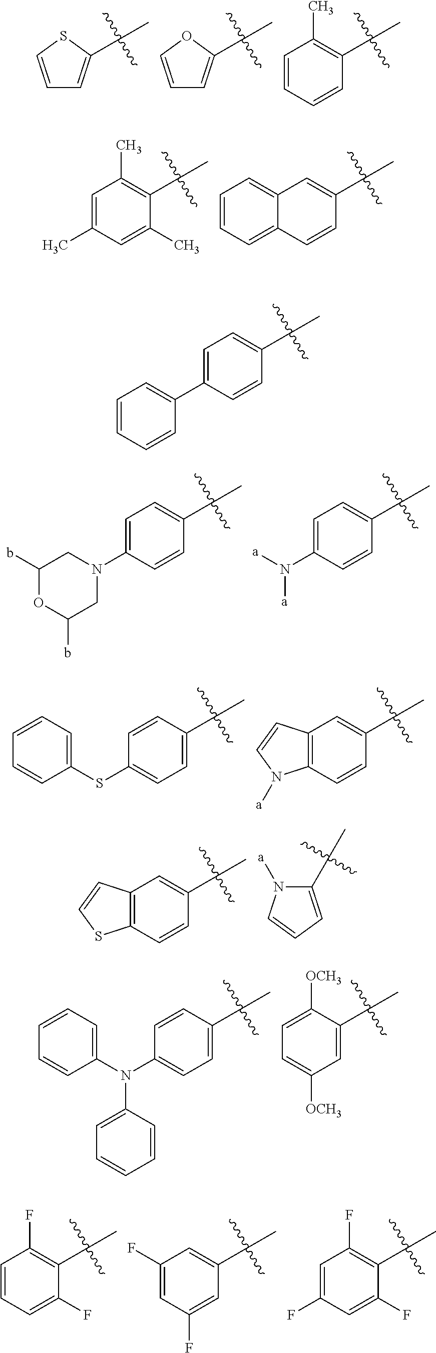

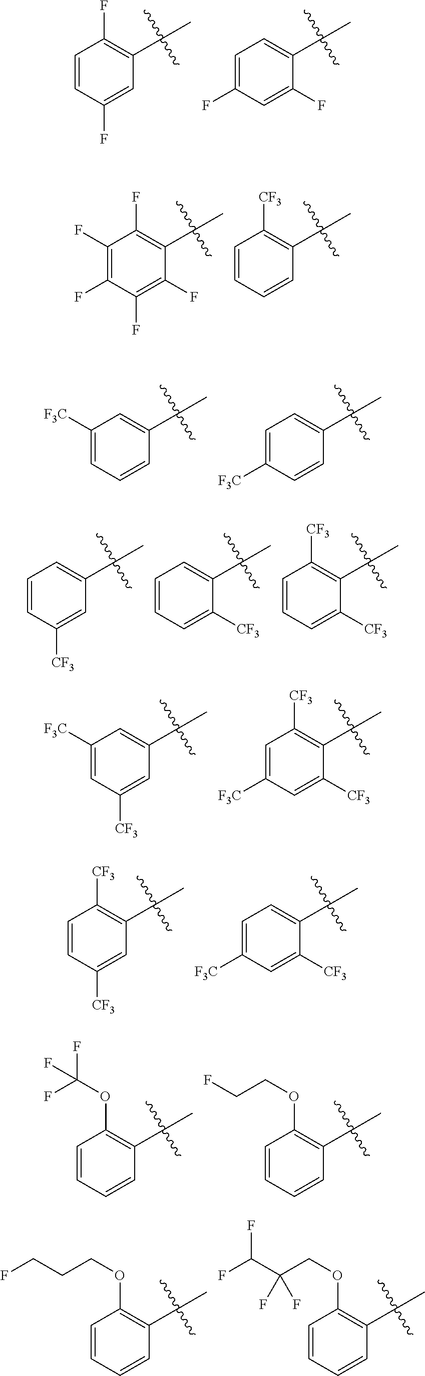

wherein each a is a methyl or ethyl group and each b is H or a methyl group.

In preferred embodiments, the α-ketoxime ester compound of Formula 1 may be represented by Formula 2:

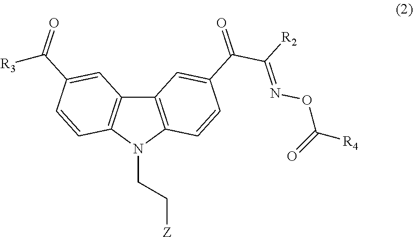

In preferred embodiments, the oxime ester compound of Formula 1 may be represented by Formula 3:

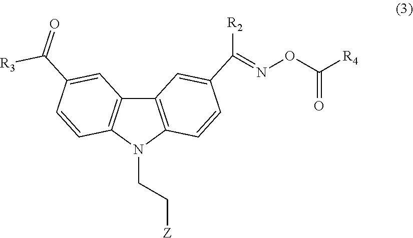

R₂ in Formulae 2 and 3 is as defined in Formula 1. R₂ is preferably methyl, tolyl, 2-methylbenzyl,

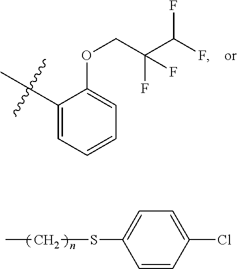

(n=1-4). R₃ is preferably thienyl, naphthyl, tolyl,

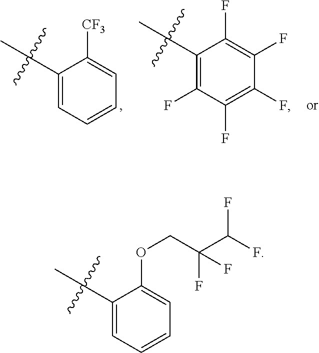 [SMILES: *c1ccccc1C(F)(F)F ; "," ; *c1c(F)c(F)c(F)c(F)c1F ; "or" ; *c1ccccc1OCC(F)(F)C(F)F]

The substituent R₃ serves to shift the UV absorption region of the initiator to a longer wavelength, achieving high sensitivity of the initiator. For example, in the case where R₃ is a thienyl group, a coplanar structure of the initiator may be formed and the presence of “S” in the thienyl group may facilitate conjugation of the initiator. R₄ is preferably methyl or phenyl. Z may be —H, —R₅, —OR₅, —OC(O)OR₅, —C(O)OR₅ or —OC(O)OR₅. Z is preferably —OR₅, —OC(O)R₅, —C(O)OR₅ or —OC(O)OR₅, more preferably —OR₅ or —OC(O)R₅, which can further improve the solubility of the initiator. R₅ may be C₁-C₆ linear, branched or cyclic alkyl,

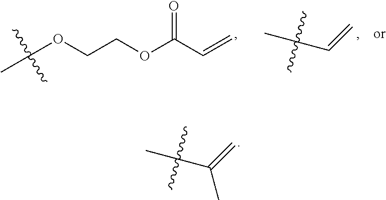

Z is preferably —OR₅ or —OC(O)R₅.

More preferably, the compound represented by Formula 2 is preferably selected from the compounds of Formulae 4-1 to 4-23:

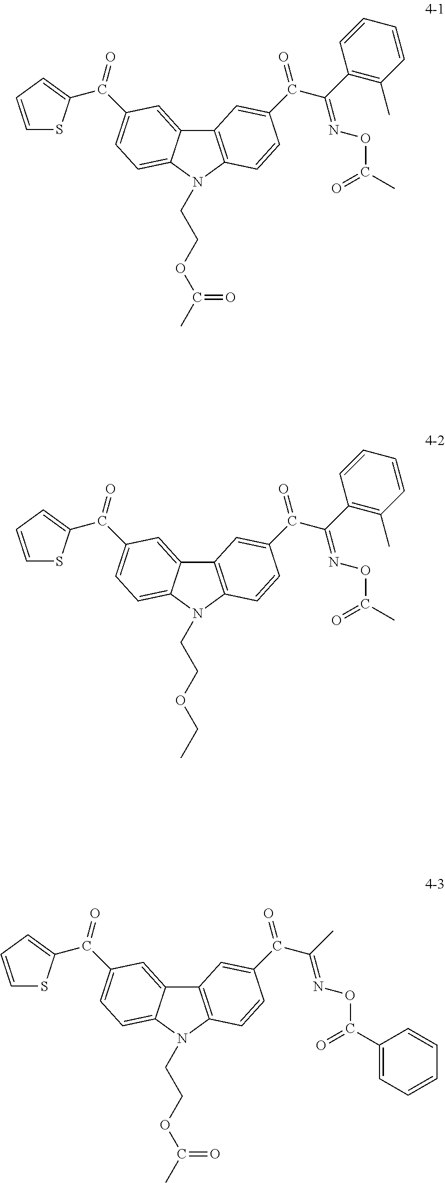 [SMILES: CC(=O)OCCn1c2ccc(C(=O)/C(=N/OC(C)=O)c3ccccc3C)cc2c2cc(C(=O)c3cccs3)ccc21 ; CCOCCn1c2ccc(C(=O)/C(=N/OC(C)=O)c3ccccc3C)cc2c2cc(C(=O)c3cccs3)ccc21 ; CC(=O)OCCn1c2ccc(C(=O)/C(C)=N/OC(=O)c3ccccc3)cc2c2cc(C(=O)c3cccs3)ccc21]

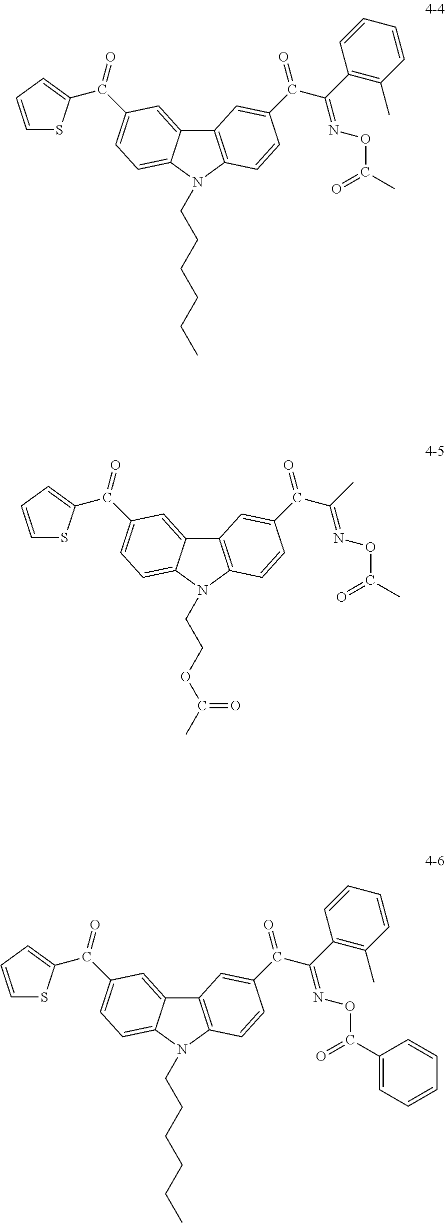 [SMILES: CCCCCCn1c2ccc(C(=O)/C(=N/OC(C)=O)c3ccccc3C)cc2c2cc(C(=O)c3cccs3)ccc21 ; CC(=O)OCCn1c2ccc(C(=O)/C(C)=N/OC(C)=O)cc2c2cc(C(=O)c3cccs3)ccc21 ; CCCCCCn1c2ccc(C(=O)/C(=N/OC(=O)c3ccccc3)c3ccccc3C)cc2c2cc(C(=O)c3cccs3)ccc21]

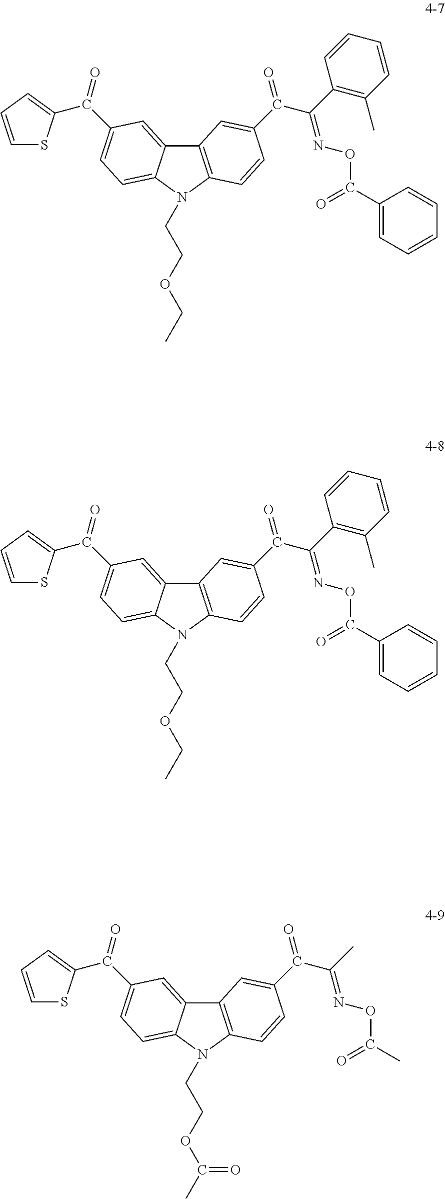 [SMILES: CCOCCn1c2ccc(C(=O)/C(=N/OC(=O)c3ccccc3)c3ccccc3C)cc2c2cc(C(=O)c3cccs3)ccc21 ; CCOCCn1c2ccc(C(=O)/C(=N/OC(=O)c3ccccc3)c3ccccc3C)cc2c2cc(C(=O)c3cccs3)ccc21 ; CC(=O)OCCn1c2ccc(C(=O)/C(C)=N/OC(C)=O)cc2c2cc(C(=O)c3cccs3)ccc21]

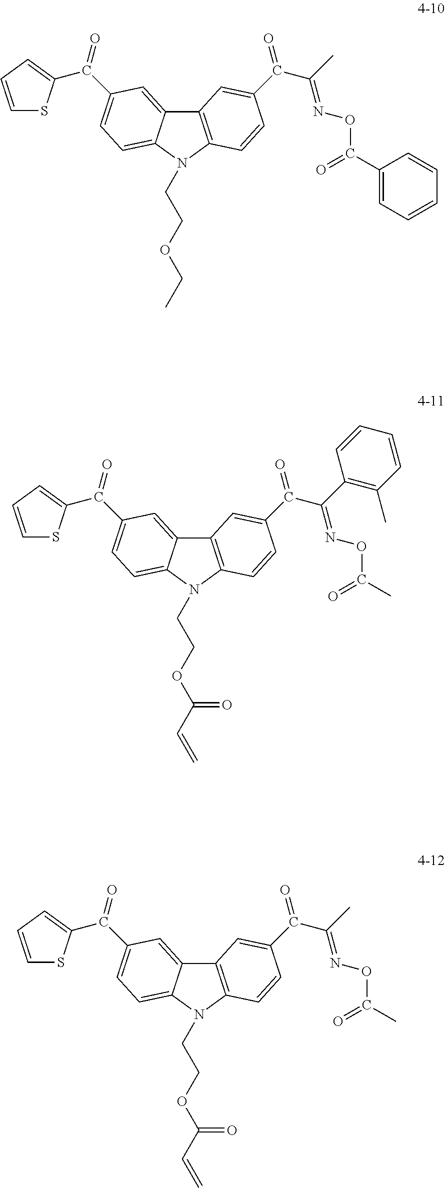 [SMILES: CCOCCn1c2ccc(C(=O)/C(C)=N/OC(=O)c3ccccc3)cc2c2cc(C(=O)c3cccs3)ccc21 ; C=CC(=O)OCCn1c2ccc(C(=O)/C(=N/OC(C)=O)c3ccccc3C)cc2c2cc(C(=O)c3cccs3)ccc21 ; C=CC(=O)OCCn1c2ccc(C(=O)/C(C)=N/OC(C)=O)cc2c2cc(C(=O)c3cccs3)ccc21]

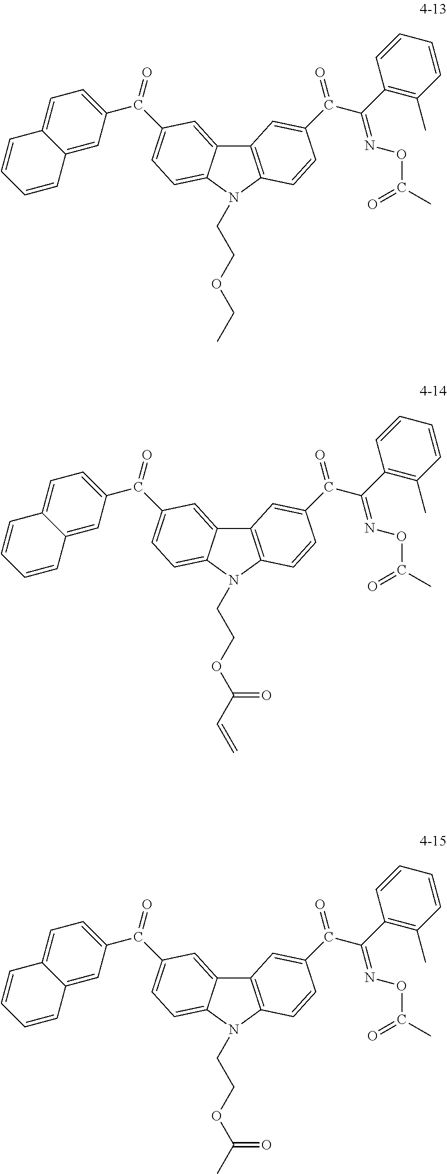 [SMILES: CCOCCn1c2ccc(C(=O)/C(=N/OC(C)=O)c3ccccc3C)cc2c2cc(C(=O)c3ccc4ccccc4c3)ccc21 ; C=CC(=O)OCCn1c2ccc(C(=O)/C(=N/OC(C)=O)c3ccccc3C)cc2c2cc(C(=O)c3ccc4ccccc4c3)ccc21 ; CC(=O)OCCn1c2ccc(C(=O)/C(=N/OC(C)=O)c3ccccc3C)cc2c2cc(C(=O)c3ccc4ccccc4c3)ccc21]

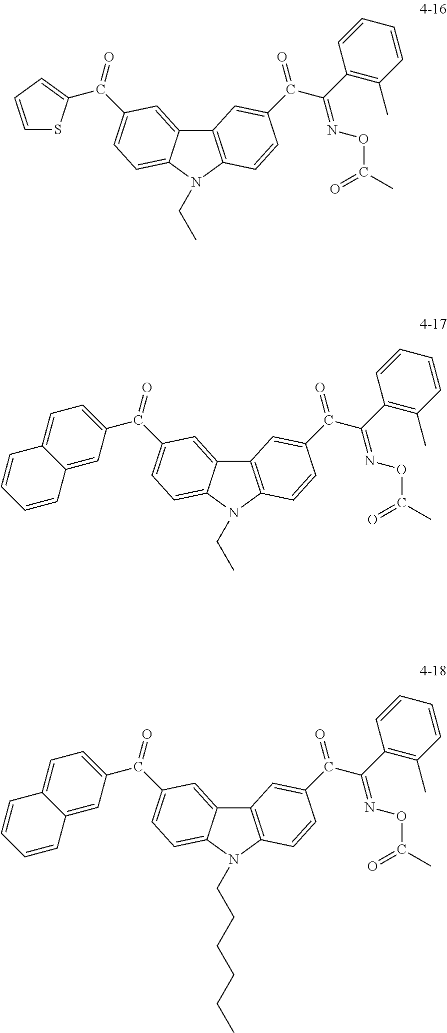 [SMILES: CCn1c2ccc(C(=O)/C(=N/OC(C)=O)c3ccccc3C)cc2c2cc(C(=O)c3cccs3)ccc21 ; CCn1c2ccc(C(=O)/C(=N/OC(C)=O)c3ccccc3C)cc2c2cc(C(=O)c3ccc4ccccc4c3)ccc21 ; CCCCCCn1c2ccc(C(=O)/C(=N/OC(C)=O)c3ccccc3C)cc2c2cc(C(=O)c3ccc4ccccc4c3)ccc21]

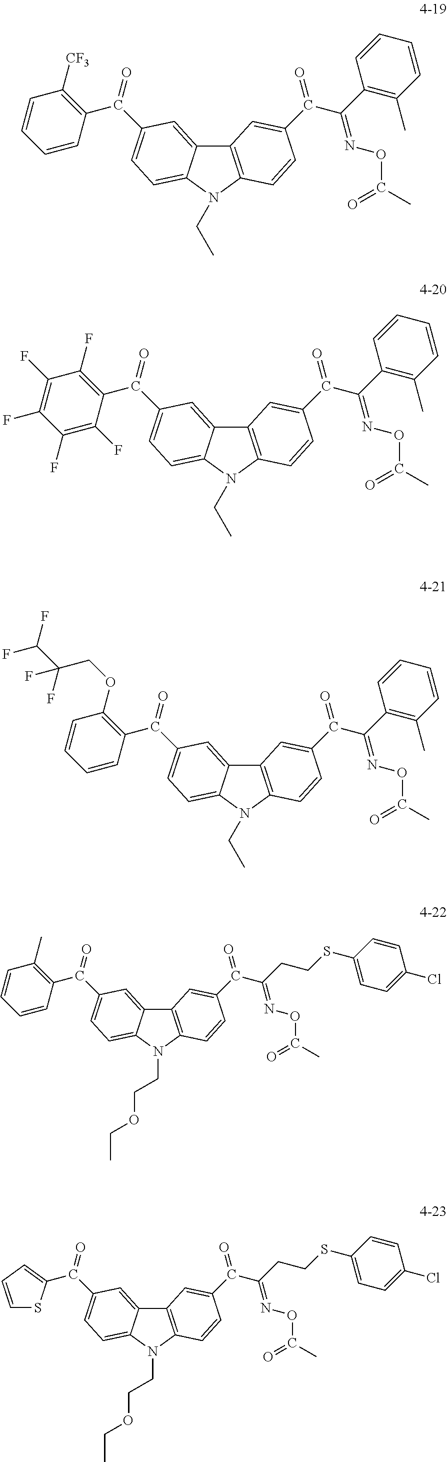 [SMILES: CCn1c2ccc(C(=O)/C(=N/OC(C)=O)c3ccccc3C)cc2c2cc(C(=O)c3ccccc3C(F)(F)F)ccc21 ; CCn1c2ccc(C(=O)/C(=N/OC(C)=O)c3ccccc3C)cc2c2cc(C(=O)c3c(F)c(F)c(F)c(F)c3F)ccc21 ; CCn1c2ccc(C(=O)/C(=N/OC(C)=O)c3ccccc3C)cc2c2cc(C(=O)c3ccccc3OCC(F)(F)C(F)F)ccc21 ; CCOCCn1c2ccc(C(=O)/C(CCSc3ccc(Cl)cc3)=N/OC(C)=O)cc2c2cc(C(=O)c3ccccc3C)ccc21 ; CCOCCn1c2ccc(C(=O)/C(CCSc3ccc(Cl)cc3)=N/OC(C)=O)cc2c2cc(C(=O)c3cccs3)ccc21]

The compound represented by Formula 3 is preferably selected from the compounds of Formulae 5-1 to 5-26:

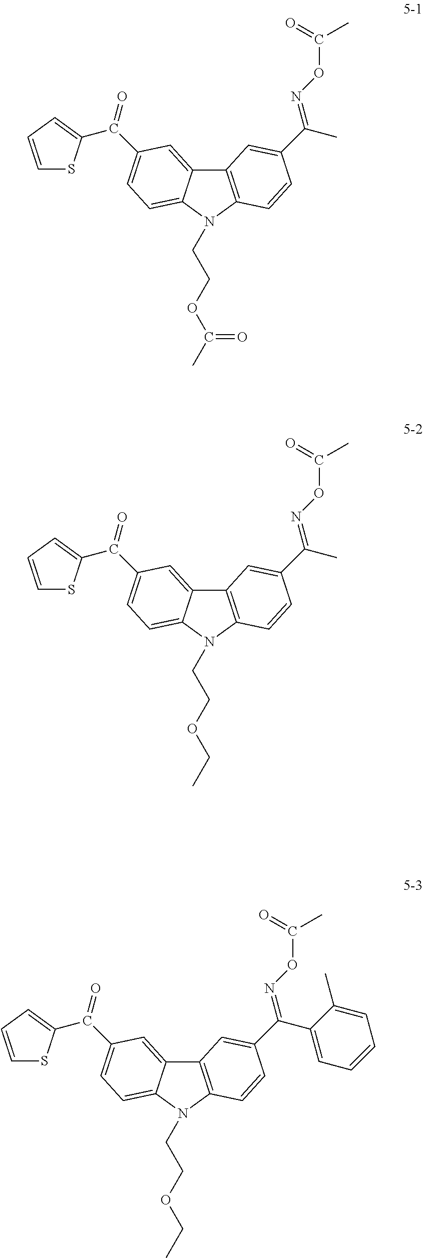 [SMILES: CC(=O)OCCn1c2ccc(C(=O)c3cccs3)cc2c2cc(/C(C)=N/OC(C)=O)ccc21 ; CCOCCn1c2ccc(C(=O)c3cccs3)cc2c2cc(/C(C)=N/OC(C)=O)ccc21 ; CCOCCn1c2ccc(C(=O)c3cccs3)cc2c2cc(/C(=N/OC(C)=O)c3ccccc3C)ccc21]

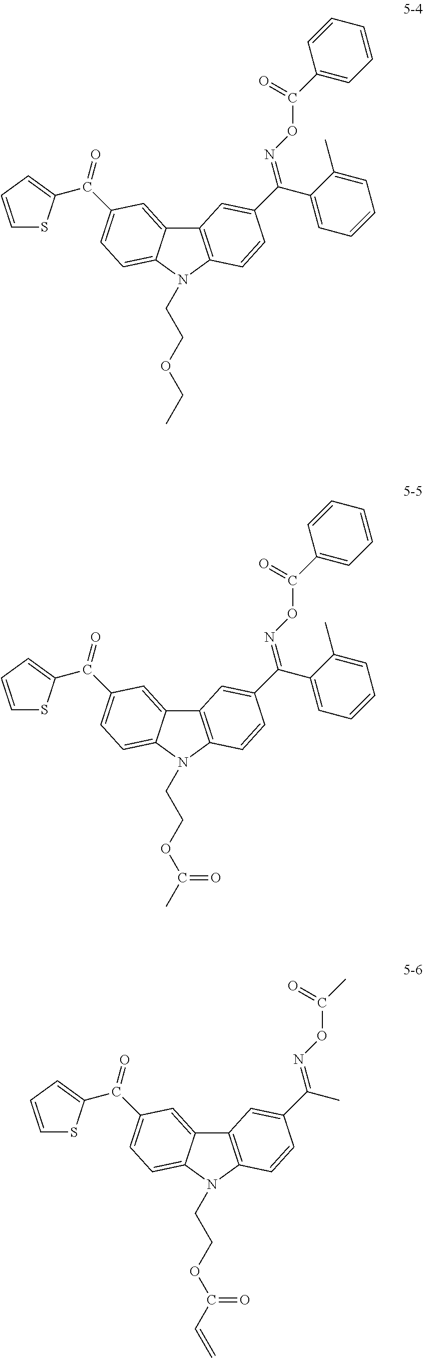 [SMILES: CCOCCn1c2ccc(C(=O)c3cccs3)cc2c2cc(/C(=N/OC(=O)c3ccccc3)c3ccccc3C)ccc21 ; CC(=O)OCCn1c2ccc(C(=O)c3cccs3)cc2c2cc(/C(=N/OC(=O)c3ccccc3)c3ccccc3C)ccc21 ; C=CC(=O)OCCn1c2ccc(C(=O)c3cccs3)cc2c2cc(/C(C)=N/OC(C)=O)ccc21]

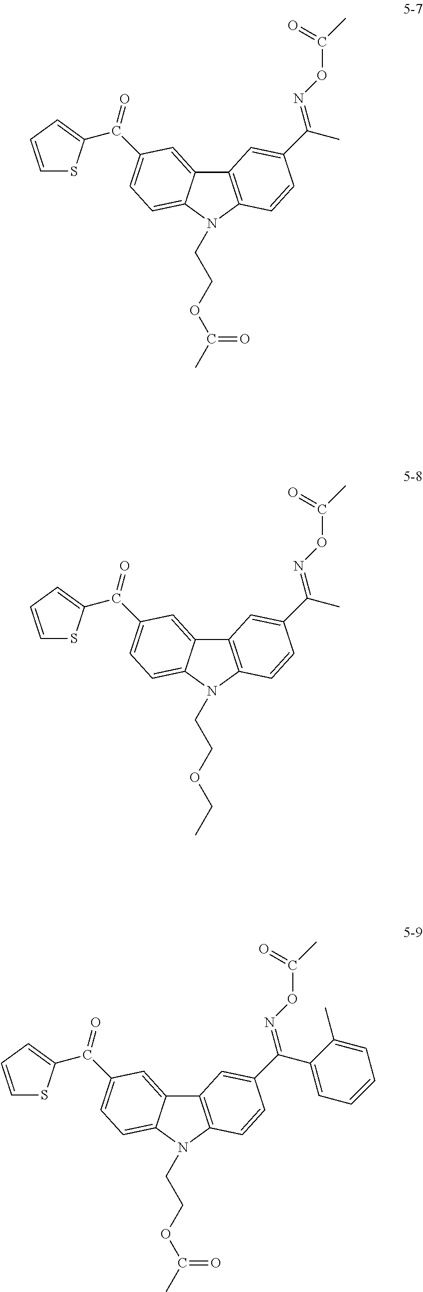 [SMILES: CC(=O)OCCn1c2ccc(C(=O)c3cccs3)cc2c2cc(/C(C)=N/OC(C)=O)ccc21 ; CCOCCn1c2ccc(C(=O)c3cccs3)cc2c2cc(/C(C)=N/OC(C)=O)ccc21 ; CC(=O)OCCn1c2ccc(C(=O)c3cccs3)cc2c2cc(/C(=N/OC(C)=O)c3ccccc3C)ccc21]

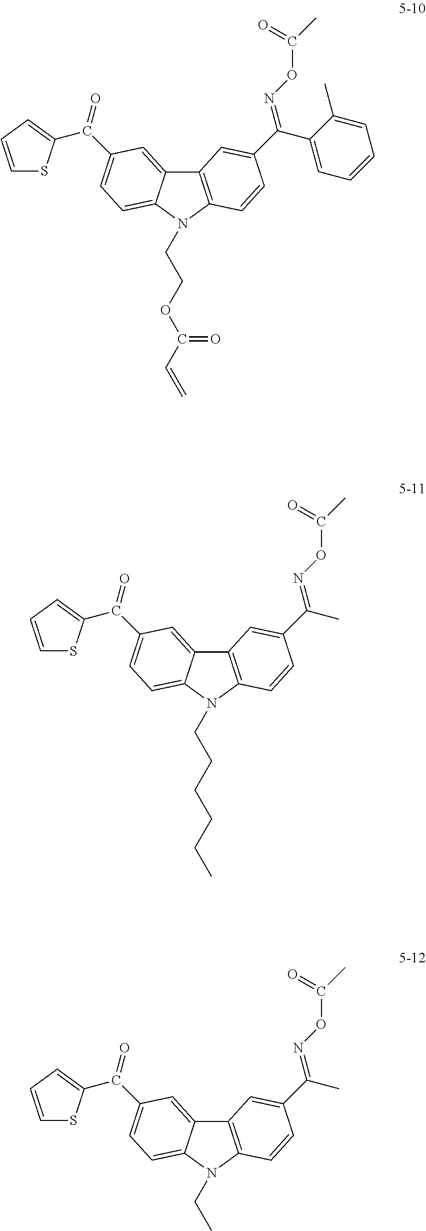 [SMILES: C=CC(=O)OCCn1c2ccc(C(=O)c3cccs3)cc2c2cc(/C(=N/OC(C)=O)c3ccccc3C)ccc21 ; CCCCCCn1c2ccc(C(=O)c3cccs3)cc2c2cc(/C(C)=N/OC(C)=O)ccc21 ; CCn1c2ccc(C(=O)c3cccs3)cc2c2cc(/C(C)=N/OC(C)=O)ccc21]

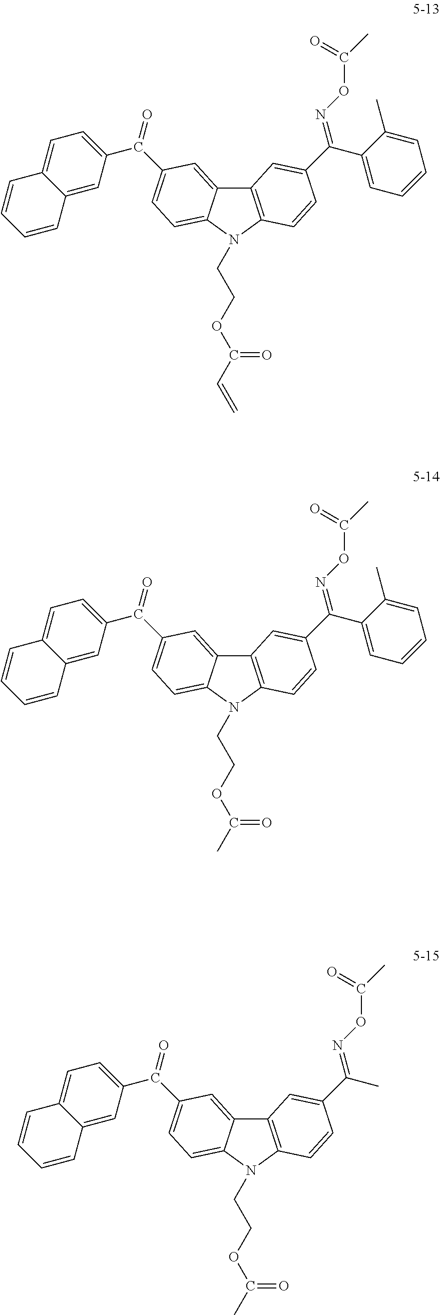 [SMILES: C=CC(=O)OCCn1c2ccc(C(=O)c3ccc4ccccc4c3)cc2c2cc(/C(=N/OC(C)=O)c3ccccc3C)ccc21 ; CC(=O)OCCn1c2ccc(C(=O)c3ccc4ccccc4c3)cc2c2cc(/C(=N/OC(C)=O)c3ccccc3C)ccc21 ; CC(=O)OCCn1c2ccc(C(=O)c3ccc4ccccc4c3)cc2c2cc(/C(C)=N/OC(C)=O)ccc21]

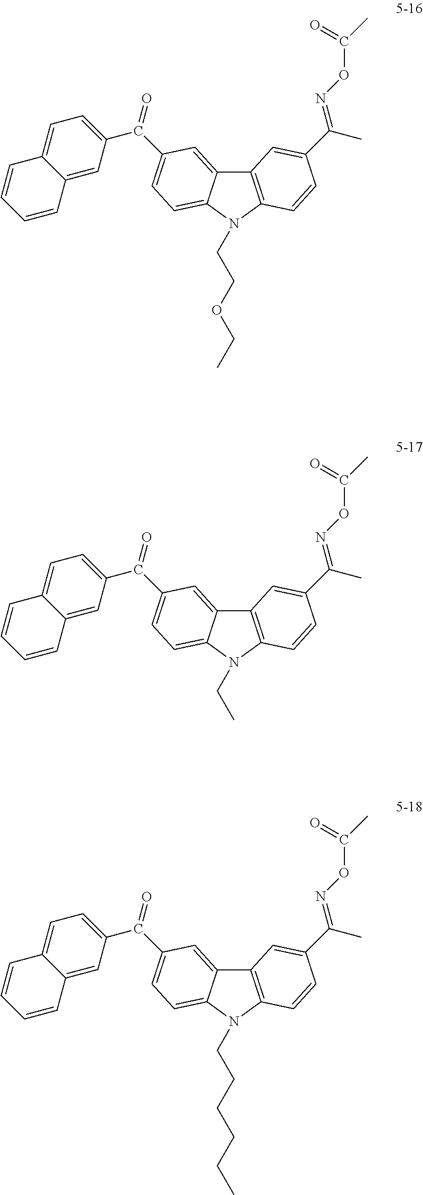 [SMILES: CCOCCn1c2ccc(C(=O)c3ccc4ccccc4c3)cc2c2cc(/C(C)=N/OC(C)=O)ccc21 ; CCn1c2ccc(C(=O)c3ccc4ccccc4c3)cc2c2cc(/C(C)=N/OC(C)=O)ccc21 ; CCCCCCn1c2ccc(C(=O)c3ccc4ccccc4c3)cc2c2cc(/C(C)=N/OC(C)=O)ccc21]

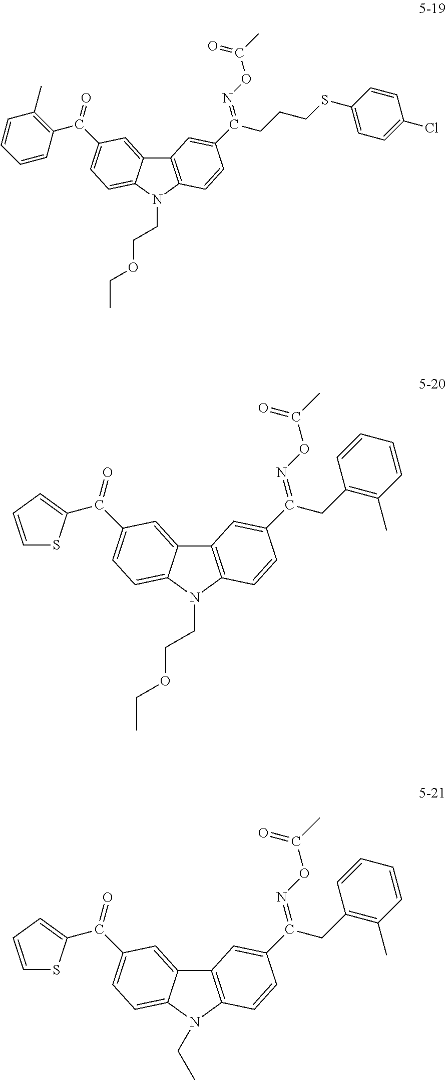 [SMILES: CCOCCn1c2ccc(C(=O)c3ccccc3C)cc2c2cc(/C(CCCSc3ccc(Cl)cc3)=N/OC(C)=O)ccc21 ; CCOCCn1c2ccc(C(=O)c3cccs3)cc2c2cc(/C(Cc3ccccc3C)=N/OC(C)=O)ccc21 ; CCn1c2ccc(C(=O)c3cccs3)cc2c2cc(/C(Cc3ccccc3C)=N/OC(C)=O)ccc21]

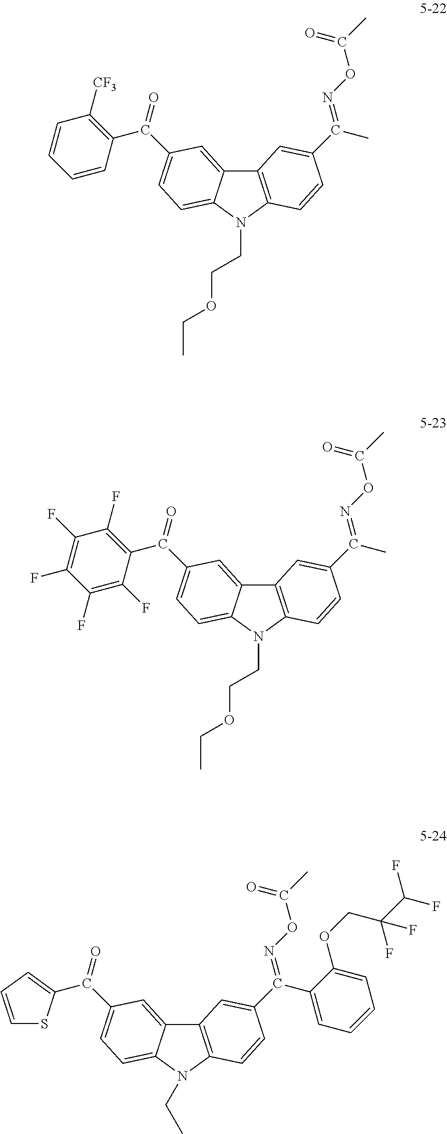 [SMILES: CCOCCn1c2ccc(C(=O)c3ccccc3C(F)(F)F)cc2c2cc(/C(C)=N/OC(C)=O)ccc21 ; CCOCCn1c2ccc(C(=O)c3c(F)c(F)c(F)c(F)c3F)cc2c2cc(/C(C)=N/OC(C)=O)ccc21 ; CCn1c2ccc(C(=O)c3cccs3)cc2c2cc(/C(=N/OC(C)=O)c3ccccc3OCC(F)(F)C(F)F)ccc21]

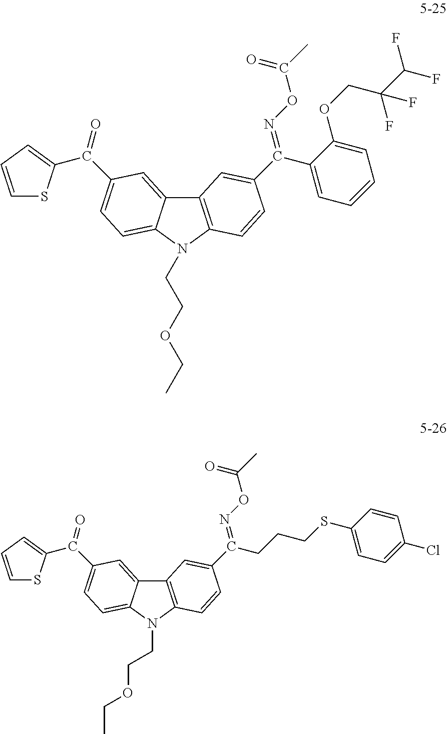 [SMILES: CCOCCn1c2ccc(C(=O)c3cccs3)cc2c2cc(/C(=N/OC(C)=O)c3ccccc3OCC(F)(F)C(F)F)ccc21 ; CCOCCn1c2ccc(C(=O)c3cccs3)cc2c2cc(/C(CCCSc3ccc(Cl)cc3)=N/OC(C)=O)ccc21]

The photoinitiator of the present invention is an oxime ester-based compound including a carbazole skeleton in which the nitrogen of the carbazole is substituted with an alkoxyalkyl or acyloxyalkyl group. Due to this structure, the photoinitiator has improved solubility and outstanding photosensitivity.

### Synthesis of the α-Ketoxime Ester Compound of Formula 2 Including Carbazole Structure

There is no restriction on the method for preparing the compound of Formula 2. For example, the compound of Formula 2 may be prepared by the synthetic route depicted in Reaction Scheme 1:

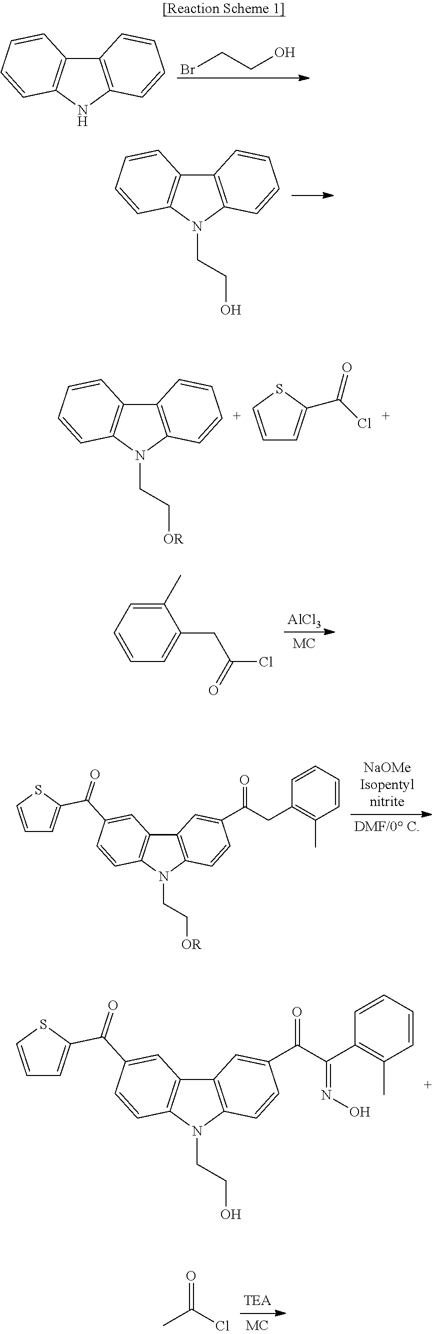

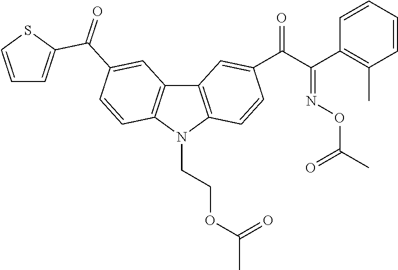 [SMILES: CC(=O)OCCn1c2ccc(C(=O)/C(=N/OC(C)=O)c3ccccc3C)cc2c2cc(C(=O)c3cccs3)ccc21]

First, a carbazole compound, thiophene carbonyl chloride, and 2-(o-tolyl)acetyl chloride are sequentially allowed to react in the presence of aluminum chloride to obtain an acyl compound. Then, the acyl compound is reacted with isoamyl nitrite in the presence of a basic catalyst to obtain the α-ketoxime compound. Next, the α-ketoxime compound is reacted with carbonyl chloride in the presence of triethylamine as a catalyst, yielding the α-ketoxime ester compound represented by Formula 2.

### Synthesis of the Oxime Ester Compound of Formula 3 Having Carbazole Structure

There is no restriction on the method for preparing the compound of Formula 3. For example, the compound of Formula 3 may be prepared by the synthetic route depicted in Reaction Scheme 2:

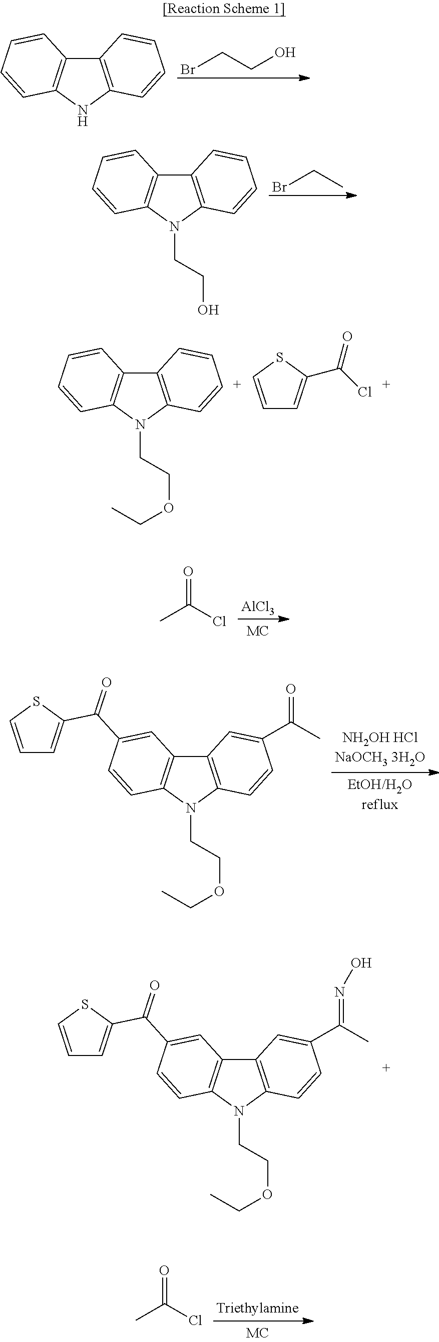

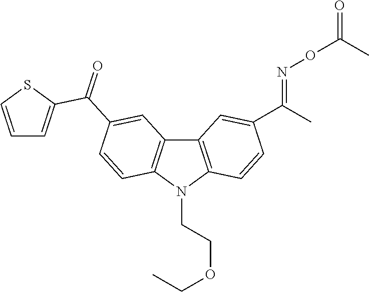 [SMILES: CCOCCn1c2ccc(C(=O)c3cccs3)cc2c2cc(/C(C)=N/OC(C)=O)ccc21]

First, the carbazole compound, thiophene carbonyl chloride, and carbonyl chloride are sequentially allowed to react in the presence of aluminum chloride to obtain the acyl compound. Then, the acyl compound is reacted with hydroxylamine in the presence of hydrochloric acid as a catalyst to obtain the oxime compound. Next, the oxime compound is reacted with carbonyl chloride in the presence of triethylamine as a catalyst, yielding the photoinitiator having an oxime ester group represented by Formula 3.

The photoinitiator of the photosensitive resin composition that is represented by Formula 1 according to the present invention may be used alone or in combinations of two or more. The photoinitiator may also be used in combination with other known photoinitiators.

When one or more different oxime ester compounds that may be represented by Formula 1 are mixed with other known photoinitiators, it is preferred that the oxime ester compounds are included in an amount of at least 50% by weight, based on the total weight of all photoinitiators. The presence of the oxime ester compounds in an amount of at least 50% by weight can increase the solubility of the photoinitiator while effectively maintaining the sensitivity of the photoinitiator.

Examples of the known photoinitiators include: acetophenones, such as acetophenone, 2,2-diethoxyacetophenone, p-dimethylacetophenone, p-dimethylaminopropiophenone, dichloroacetophenone, trichloroacetophenone, and p-tert-butylacetophenone; benzophenones, such as benzophenone, 2-chlorobenzophenone, and p,p′-bisdimethylaminobenzophenone; benzoin ethers, such as benzil, benzoin, benzoin methyl ether, benzoin isopropyl ether, and benzoin isobutyl ether; sulfur compounds, such as benzil dimethyl ketal, thioxanthene, 2-chlorothioxanthene, 2,4-diethylthioxanthene, 2-methylthioxanthene, and 2-isopropylthioxanthene; anthraquinones, such as 2-ethylanthraquinone, octamethylanthraquinone, 1,2-benzanthraquinone, and 2,3-diphenylanthraquinone; organic peroxides, such as azobisisobutyronitrile, benzoyl peroxide, and cumene peroxide; thiol compounds, such as 2-mercaptobenzimidazole, 2-mercaptobenzoxazole, and 2-mercaptobenzothiazole; imidazolyl compounds, such as 2-(o-chlorophenyl)-4,5-di(m-methoxyphenyl)-imidazolyl dimer; triazine compounds, such as p-methoxytriazine; triazine compounds having a halomethyl group, such as 2,4,6-tris(trichloromethyl)-s-triazine, 2-methyl-4,6-bis(trichloromethyl)-s-triazine, 2-[2-(5-methylfuran-2-yl)ethenyl]-4,6-bis(trichloromethyl)-s-triazine, 2-[2-(furan-2-yl)ethenyl]-4,6-bis(trichloromethyl)-s-triazine, 2-[2-(4-diethylamino-2-methylphenyl)ethenyl]-4,6-bis(trichloromethyl)-s-triazine, 2-[2-(3,4-dimethoxyphenol)ethenyl]4,6-bis(trichloromethyl)-s-triazine, 2-(4-methoxyphenyl)-4,6-bis(trichloromethyl)-s-triazine, 2-(4-ethoxystyryl)-4,6-bis(trichloromethyl)-s-triazine, and 2-(4-n-butoxyphenyl)-4,6-bis(trichloromethyl)-s-triazine; and amino ketone compounds, such as 2-benzyl-2-dimethylamino-1-(4-morpholinophenyl)-butan-1-one.

The photosensitive resin composition of the present invention may further include a sensitizer. Examples of suitable sensitizers include: cationic dyes, such as cyanine, xanthene, oxazine, thiazine, diarylmethane, triarylmethane, and pyrylium dyes; neutral dyes, such as merocyanine, coumarin, indigo, aromatic amine, phthalocyanine, azo, quinone, and thioxanthene sensitizing dyes; and other compounds, such as benzophenones, acetophenones, benzoins, thioxanthones, anthraquinones, imidazoles, biimidazoles, coumarins, ketocoumarines, triphenylpyryliums, triazines, and benzoic acids.

The photosensitive resin composition of the present invention may further include a solvent, a polymeric compound soluble in an aqueous alkaline solution, or a mixture of the polymeric compound and a photopolymerizable compound having an ethylenically unsaturated bond. Specific examples of suitable solvents, polymeric compounds soluble in an aqueous alkaline solution, and photopolymerizable compounds having an ethylenically unsaturated bond include: monomers and oligomers, such as acrylic acid, methacrylic acid, fumaric acid, maleic acid, monomethyl fumarate, monoethyl fumarate, 2-hydroxyethyl acrylate, 2-hydroxyethyl methacrylate, ethylene glycol monomethyl ether acrylate, ethylene glycol monomethyl ether methacrylate, ethylene glycol monoethyl ether acrylate, ethylene glycol monoethyl ether methacrylate, glycerol acrylate, glycerol methacrylate, acrylamide, methacrylamide, acrylonitrile, methacrylonitrile, methyl acrylate, methyl methacrylate, ethyl acrylate, ethyl methacrylate, isobutyl acrylate, isobutyl methacrylate, 2-ethylhexyl acrylate, 2-ethylhexyl methacrylate, benzyl acrylate, benzyl methacrylate, ethylene glycol diacrylate, ethylene glycol dimethacrylate, diethylene glycol diacrylate, triethylene glycol diacrylate, triethylene glycol dimethacrylate, tetraethylene glycol diacrylate, tetraethylene glycol dimethacrylate, butylene glycol dimethacrylate, propylene glycol diacrylate, propylene glycol dimethacrylate, trimethylolpropane triacrylate, trimethylolpropane trimethacrylate, tetramethylolpropane tetraacrylate, tetramethylolpropane tetramethacrylate, pentaerythritol triacrylate, pentaerythritol trimethacrylate, pentaerythritol tetraacrylate, pentaerythritol tetramethacrylate, dipentaerythritol pentaacrylate, dipentaerythritol pentamethacrylate, dipentaerythritol hexaacrylate, dipentaerythritol hexamethacrylate, 1,6-hexanediol diacrylate, 1,6-hexanediol dimethacrylate, and cardoepoxy diacrylate; polyester (meth)acrylates obtained by reaction of (meth)acrylic acid with a polyester prepolymer as a condensation product of a polyhydric alcohol and a monobasic or polybasic acid; polyurethane (meth)acrylates obtained by reaction of (meth)acrylic acid with a reaction product of a compound having a polyol group and a compound having two isocyanate groups; and epoxy (meth)acrylate resins obtained by reaction of (meth)acrylic acid with an epoxy resin, such as a bisphenol A epoxy resin, a bisphenol F epoxy resin, a bisphenol S epoxy resin, a phenol or cresol novolac epoxy resin, a resol epoxy resin, a triphenolmethane epoxy resin, a polycarboxylate polyglycidyl ester, a polyol polyglycidyl ester, an aliphatic or alicyclic epoxy resin, an amine epoxy resin, or a dihydroxybenzene epoxy resin. Resins obtained by reaction of the epoxy (meth)acrylate resins with a polybasic anhydride may also be used. These photopolymerizable compounds may also be cardo-based resins.

Particularly, the solvent or the polymer soluble in an aqueous alkaline solution is a highly transparent high-molecular-weight polymer and is soluble in a developing solution (a solvent or an aqueous alkaline solution). Examples of such high-molecular-weight polymers include thermosetting resins, thermoplastic resins, and photosensitive resins, which may be used alone or as a mixture of two or more thereof. High-molecular-weight polymers with good resistance to heat, solvents, and chemicals are particularly preferred.

The use of a polyfunctional (meth)acrylic monomer as the compound having an ethylenically unsaturated bond is advantageous in terms of sensitivity to light exposure and resistance to various factors after curing.

The photosensitive resin composition of the present invention can be applied to a resist for the production of a color filter and a black matrix. In this case, the photosensitive resin composition of the present invention may contain a pigment or colorant.

Examples of suitable colorants include red, green, and blue colorants, and cyan, magenta, yellow, and black pigments of subtractive color-mixing systems. Examples of suitable pigments include C.I. Pigment Yellow 12, 13, 14, 17, 20, 24, 55, 83, 86, 93, 109, 110, 117, 125, 137, 139, 147, 148, 153, 154, 166, and 168, C.I. Pigment Orange 36, 43, 51, 55, 59, and 61, C.I. Pigment Red 9, 97, 122, 123, 149, 168, 177, 180, 192, 215, 216, 217, 220, 223, 224, 226, 227, 228, and 240, C.I. Pigment Violet 19, 23, 29, 30, 37, 40, and 50, C.I. Pigment Blue 15, 15:1, 15:4, 15:6, 22, 60, and 64, C.I. Pigment Green 7 and 36, C.I. Pigment Brown 23, 25, and 26, C.I. Pigment Black 7, and titan black.

The present invention also provides a column spacer, a black matrix, a color filter, or a substrate having an organic insulating film produced using the photosensitive resin composition. The present invention also provides a substrate having a film formed by coating the photosensitive resin composition. The film may be used in the surface of a plasma display panel or a polarizing plate for a liquid crystal display. The film may also be used in various applications, including sunglass lenses, glass lenses with power, finder lenses for cameras, instrument covers, automobile windows, tram windows, brightness enhancement films, and optical waveguide films.

The photosensitive composition of the present invention may be used to form a pattern by the following procedure. Specifically, the photosensitive composition of the present invention is applied to a substrate, volatiles such as a solvent are removed from the photosensitive composition layer, the layer from which the volatiles have been removed is exposed to light through a photomask, and the exposed layer is developed to form a pattern. Thus, the present invention provides a cured film obtained by the above curing procedure.

The substrate may be, for example, a glass substrate, a silicon substrate, a polycarbonate substrate, a polyester substrate, an aromatic polyamide substrate, a polyamideimide substrate, a polyimide substrate, an aluminum substrate, a GaAs substrate, etc.

There is no restriction on the method for applying the photosensitive resin composition to the substrate. For example, the photosensitive resin composition may be applied to the substrate by any suitable technique known in the art, such as spin coating, casting, roll coating, or slit & spin coating. The photosensitive resin composition may also be applied using any suitable means known in the art, such as a spinless coater.

Subsequently, the photosensitive composition layer is heated to remove volatiles such as a solvent. As a result, a layer composed of the solid components of the photosensitive composition is formed on the substrate. Then, the layer is exposed to light. For example, the layer may be selectively exposed to active energy rays through a photomask. A low pressure mercury lamp, a medium pressure mercury lamp, a high pressure mercury lamp, an ultra-high pressure mercury lamp, a xenon lamp, or a metal halogen lamp is generally suitable as the exposure light source. For example, laser light may also be used as the active energy source for exposure. Other light sources, such as electron rays, α-rays, β-rays, γ-rays, X-rays, and neutron rays may also be used. The active energy rays are irradiated to the layer through a photomask. For example, the photomask has a structure in which a light-blocking layer is disposed on the surface of a glass plate to shield the incident active energy rays. A region in which the light-blocking layer is not formed on the glass plate is a light transmission region. As a result of the exposure, the photosensitive composition layer is divided into two regions: a region onto which the active energy rays are not irradiated and a region onto which the active energy rays are irradiated. The exposed layer has a pattern corresponding to the pattern of the light transmission region.

The substrate having undergone light exposure is developed with a suitable developing solution, for example, a dilute aqueous alkaline solution. For example, the development may be performed in such a manner that the photosensitive composition layer having undergone light exposure is brought into contact with a dilute aqueous alkaline solution. Specifically, the substrate, on which the photosensitive composition layer is formed, is dipped in or showered with a dilute aqueous alkaline solution. The dilute aqueous alkaline solution may be, for example, an aqueous solution of an alkaline compound, such as sodium carbonate, potassium carbonate, sodium hydroxide, potassium hydroxide, tetramethylammonium hydroxide or an organic amine As a result of the development, the unirradiated region of the photosensitive composition layer is removed. The irradiated region remains unremoved and forms the pattern.

The substrate having undergone development is washed with water and dried to form the desired pattern. The washing and drying are performed by techniques known in the art.

The present invention will be explained in more detail with reference to the following examples. However, these examples are provided for illustrative purposes and are not intended to limit the scope of the invention.

### EXAMPLE 1

### Synthesis of the Compound of Formula 4-1

### 1 Step: Synthesis of 1-(9-(2-hydroxyethyl)-6-(thiophene-2-carbonyl)-9H-carbazol-3-yl)-2-(o-tolyl)ethanone

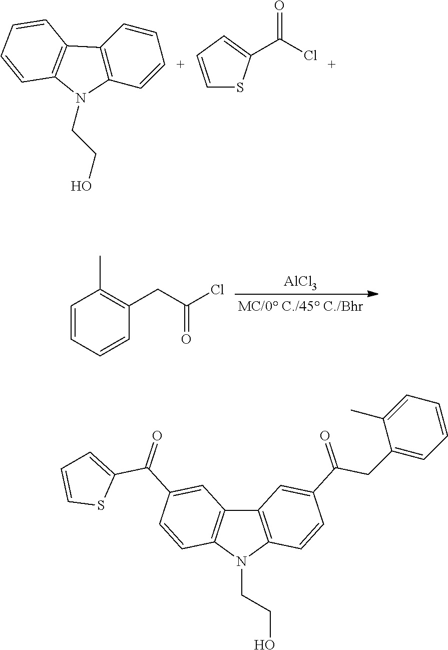 [SMILES: OCCn1c2c(c3ccccc31)C=C[C+]=C2 ; O=C(Cl)c1cccs1 ; Cc1ccccc1CC(=O)Cl ; "AlCl3" ; Cc1ccccc1CC(=O)c1ccc2c(c1)c1cc(C(=O)c3cccs3)ccc1n2CCO]

100.0 g of 2-(9H-carbazol-9-yl)ethanol was dissolved in 600 mL of dry dichloromethane. The solution was cooled to 0° C., and then 66.3 g of AlCl₃ was added thereto. To the mixture was slowly added 73.2 g of thiophene carbonylchloride at 5° C. The mixture was stirred at 25° C. for 8 h. After 66.3 g of AlCl₃ was added. To the mixture was added dropwise 84.3 g of 2-(o-tolyl)acetyl chloride at 0° C. The reaction mixture was stirred at 25° C. for 7 h. After cooling to 0° C., the solution of the reactor was slowly added to 1200 mL of ice-water for layer separation, and washed with 1200 mL water. The organic layer was dried over anhydrous MgSO₄, concentrated, and purified by column chromatography, giving 120 g (yield; 56%) of the 1-(9-(2-hydroxyethyl)-6-(thiophene-2-carbonyl)-9H-carbazol-3-yl)-2-(o-tolyl)ethanone.

¹H-NMR (δ, ppm, CDCl₃): 2.35 (t, 3H), 3.61 (t, 2H), 3.65 (s, 1H,OH), 4.16 (s, 2H), 4.48 (t, 2H), 7.14-7.27 (m, 2H), 7.34 (t, 1H), 7.40 (m, 2H), 7.61-7.66 (m, 2H), 7.92 (d, 1H), 8.00 (d, 1H), 8.09-8.14 (m, 2H), 8.65 (s, 1H), 8.84 (s, 1H)

### 2 Step: Synthesis of (E)-1-(9-(2-hydroxyethyl)-6-(thiophene-2-carbonyl)-9H-carbazol-3-yl)-2-(hydroxyimino)-2-(o-tolyl)ethanone

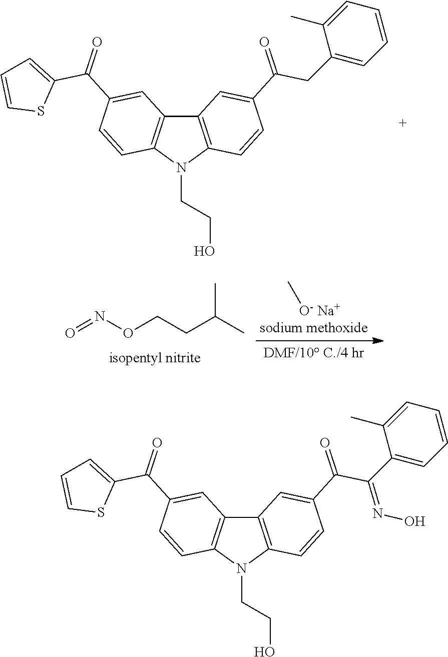

600 mL of dimethylformamide was placed in a reactor, and 100 g of the 1-(9-(2-hydroxyethyl)-6-(thiophene-2-carbonyl)-9H-carbazol-3-yl)-2-(o-tolyl)ethanone was dissolved therein. 5.95 g of sodium methoxide was added at a temperature of 25° C. To the mixture was added dropwise 27.5 g of isopentyl nitrite at 25° C. After the dropwise addition was finished, the resulting mixture was followed by stirring for 4 h. 600 mL of ethyl acetate and 600 mL of distilled water were added to wash the reaction mixture. The organic layer was dried over anhydrous MgSO₄ and concentrated. Recrystallization of the mixture with methanol and methylene chloride gave 80 g of the (E)-1-(9-(2-hydroxyethyl)-6-(thiophene-2-carbonyl)-9H-carbazol-3-yl)-2-(hydroxyimino)-2-(o-tolyl)ethanone as a yellow crystal (yield; 75%).

¹H-NMR (δ, ppm, DMSOd₆): 2.0 (s, 1H, OH), 2.48 (t, 3H), 3.63 (t, 2H), 3.8 (s, 1H, OH)), 4.47 (t, 2H), 7.23-7.33 (m, 4H), 7.56 (d, 1H), 7.71-7.74 (m, 2H), 7.92 (d, 1H), 8.01 (d, 1H), 8.10-8.16 (m, 2H), 8.60 (s, 1H), 8.85 (s, 1H)

### 3 Step: Synthesis of (E)-2-(3-(2-(acetoxyimino)-2-(o-tolyl)acetyl)-6-(thiophene-2-carbonyl)-9H-carbazol-9-yl)ethyl acetate

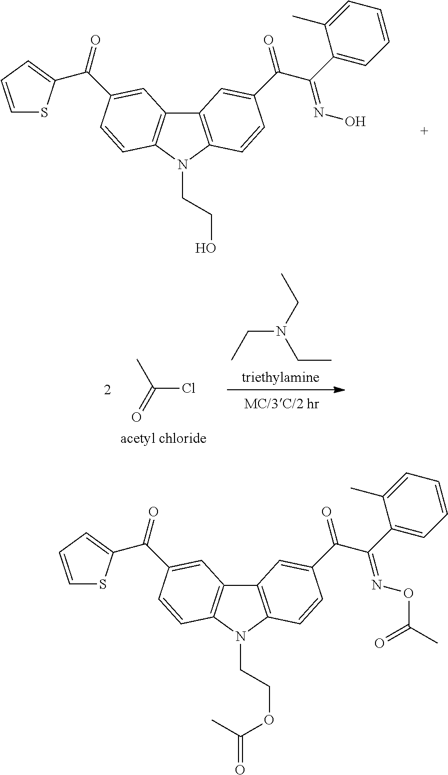

50 g of the (E)-1-(9-(2-hydroxyethyl)-6-(thiophene-2-carbonyl)-9H-carbazol-3-yl)-2-(hydroxyimino)-2-(o-tolyl)ethanone dissolved in 300 mL of dichloromethane, and 21.3 g of triethylamine was added and 16.5 g of acetyl chloride in 32 mL of dichloromethane was added dropwise thereto at 0° C. The mixture was allowed to react at 3° C. for 3 h. 400 mL of water was added to the reaction mixture to wash the organic layer twice. The organic layer was dried over anhydrous MgSO₄, and concentrated. The concentrate was recrystallized from a mixture of ethyl acetate and hexane, followed by filtration to give 45 g of the (E)-2-(3-(2-(acetoxyimino)-2-(o-tolyl)acetyl)-6-(thiophene-2-carbonyl)-9H-carbazol-9-yl)ethyl acetate as a pale yellow solid (yield; 77%).

¹H-NMR (δ, ppm, CDCl₃): 2.21 (s, 3H), 2.28 (s, 3H), 2.48 (s, 3H), 4.57 (d, 4H), 7.23-7.33 (m, 4H), 7.56 (d, 1H), 7.71-7.74 (m, 2H), 7.92 (d, 1H), 8.00 (d, 1H), 8.09 (d, 1H), 8.14 (d, 1H), 8.60 (s, 1H), 8.85 (s, 1H)

### Synthesis of the Compound of Formula 4-2

### 1 Step: Synthesis of 9-(2-ethoxyethyl)-9H-carbazole

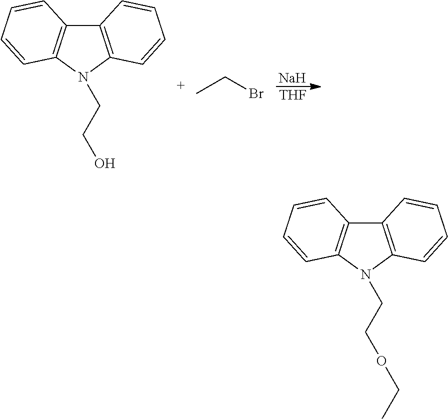

600 ml of tetrahydrofuran was placed in a 300 ml reactor, 94.7 g of NaH was added at 0° C., and a solution of 250.0 g of 2-(9H-carbazol-9-yl)ethanol in 300 ml of tetrahydrofuran was added. Following stirring at 40° C. for 15 h, 99.2 mL of bromoethane was added at 5° C. The mixture was reflux for 27 h. 350 ml of dichloromethane was added followed by concentration. After cooling, 900 ml of water and 900 ml of dichloromethane were added for washing and extraction. The organic layer was concentrated and recrystallized from ethanol, giving 218.5 g (yield; 77%) of the 9-(2-ethoxyethyl)-9H-carbazole as a solid.

¹H-NMR (δ, ppm, CDCl₃): 1.1 (t, 3H), 3.4 (q, 2H), 3.8 (t, 2H), 4.5 (t, 2H), 7.3 (m, 2H), 7.5 (q, 4H), 8.11 (s, 1H), 8.12 (s, 1H).

### 2 Step: Synthesis of (9-(2-ethoxyethyl)-9H-carbazol-3-yl)(thiophen-2-yl)methanone

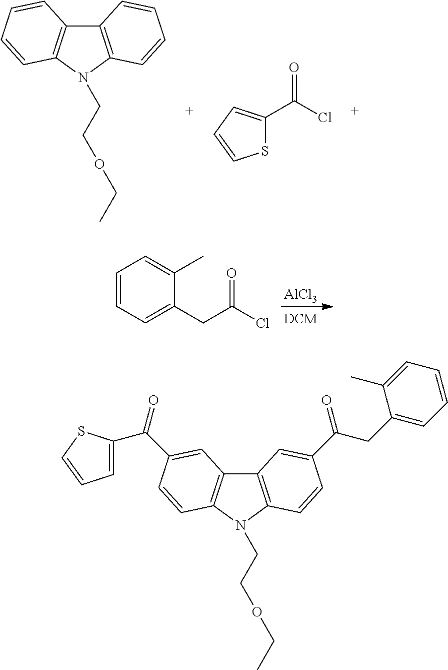

400 g of the 9-(2-ethoxyethyl)-9H-carbazole and 2.0 L of dichloromethane were placed in a 5 L round-bottomed flask equipped with a thermometer, and 240 g of aluminum chloride was added at 0° C. To the mixture was added 195 ml of 2-thiophene carbonyl chloride in 184 ml of dichloromethane. Following stirring for 6 h, the reaction was stopped. The reaction mixture was lowered to 0° C. and 240 g of aluminum chloride was added to the reaction mixture and 305 g of 2-tolyl acetyl chloride was added dropwise thereto. Following stirring for 24 h, to the organic layer was added 10 L of ice-water. The organic layer was washed twice with water. The organic layer was concentrated, dissolved in acetone, and recrystallized, giving 645 g (yield; 80%) of the (9-(2-ethoxyethyl)-9H-carbazol-3-yl) (thiophen-2-yl)methanone.

¹H-NMR (CDCl₃): δ=1.1 (t, 3H), 2.3 (s, 3H), 3.84 (q, 2H), 3.86 (t, 2H), 4.44 (s, 2H), 4.54 (t, 2H), 7.1-7.3 (m, 4H), 7.72 (m, 3H), 7.75 (t, 2H), 8.21-8.23 (dd, 2H), 8.73 (dd, 1H), 8.84 (dd, 1H).

### 3 Step: Synthesis of 1-(9-(2-ethoxyethyl)-6-(thiophene-2-carbonyl)-9H-carbazol-3-yl)-2-(hydroxyimino)-2-(o-tolyl)ethanone

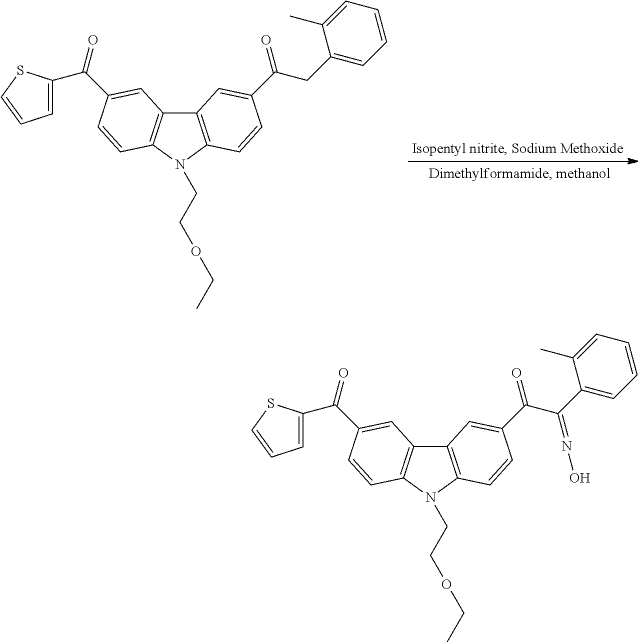 [SMILES: CCOCCn1c2ccc(C(=O)Cc3ccccc3C)cc2c2cc(C(=O)c3cccs3)ccc21 ; CCOCCn1c2ccc(C(=O)C(=NO)c3ccccc3C)cc2c2cc(C(=O)c3cccs3)ccc21]

412 g of the (9-(2-ethoxyethyl)-9H-carbazol-3-yl)(thiophen-2-yl)methanone was dissolved in 600 ml of dimethylformamide The solution was placed in a 10 L round-bottomed flask, and 3.5 L of methanol was added thereto. 145 mL of isopentyl nitrite was slowly added at 0° C. and 80.3 g of sodium methoxide was added. Following stirring at 25° C. for 47 h, the reaction was stopped. To the organic layer was added 1 L of water. Thereafter, the organic layer was washed three times with water. The organic layer was concentrated and trituration with ethyl acetate/n-hexane gave 320 g (yield; 73%) of the 1-(9-(2-ethoxyethyl)-6-(thiophene-2-carbonyl)-9H-carbazol-3-yl)-2-(hydroxyimino)-2-(o-tolyl)ethanone as a solid.

¹H-NMR (δ, ppm, CDCl₃): 1.1 (t, 3H), 2.3 (s, 3H), 3.84 (q, 2H), 3.86 (t, 2H), 4.54 (t, 2H), 7.1-7.3 (m, 4H), 7.72 (m, 3H), 7.75 (t, 2H), 8.21-8.23 (dd, 2H), 8.73 (dd, 1H), 8.84 (dd, 1H), 11 (s, 1H).

### 4 Step: Synthesis of (E)-2-(acetoxyimino)-1-(9-(2-ethoxyethyl)-6-(thiophene-2-carbonyl)-9H-carbazol-3-yl)-2-(o-tolyl)ethanone

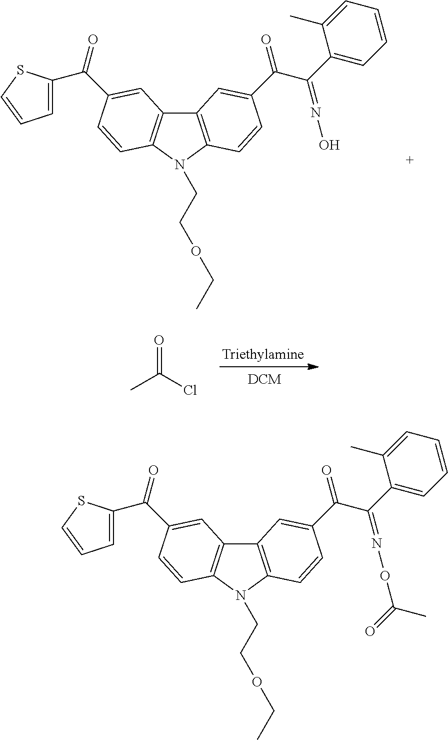

200 g of the 1-(9-(2-ethoxyethyl)-6-(thiophene-2-carbonyl)-9H-carbazol-3-yl)-2-(hydroxyimino)-2-(o-tolyl)ethanone and 2 L of dichloromethane were placed in a 5 L round-bottomed flask, and 46.5 g of triethylamine was added thereto at 5° C. A dilute solution of 35.8 g of acetyl chloride in 30 g of dichloromethane was added dropwise. Following stirring for 2 h, the organic layer was washed three times with water, concentrated. The organic layer was concentrated and trituration with ethyl acetate/n-hexane gave 180 g (yield; 83%) of the (E)-2-(acetoxyimino)-1-(9-(2-ethoxyethyl)-6-(thiophene-2-carbonyl)-9H-carbazol-3-yl)-2-(o-tolyl)ethanone as a solid.

¹H-NMR (δ, ppm, CDCl₃): 1.1 (t, 3H), 2.15 (s, 3H), 2.35 (s, 3H), 3.44 (q, 2H), 3.86 (t, 2H), 4.54 (t, 2H), 7.1-7.3 (m, 4H), 7.72 (m, 3H), 7.75 (t, 2H), 8.21-8.23 (dd, 2H), 8.73 (dd, 1H), 8.84 (dd, 1H).

### Synthesis of the Compound of Formula 4-19

### 1 Step: Synthesis of 1-(9-(2-hydroxyethyl)-6-(thiophene-2-carbonyl)-9H-carbazol-3-yl)-2-(o-tolyl)ethanone

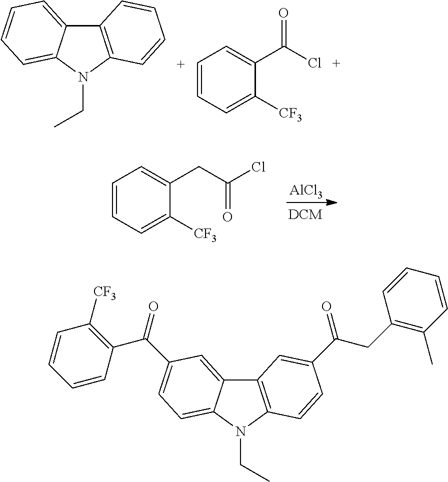

20.0 g of ethylcarbazole was dissolved in 140 mL of dry dichloromethane in a reactor. The solution was cooled to 0° C., and then 16.8 g of AlCl₃ was added thereto. To the mixture was added dropwise 25.0 g of 2-trifluoromethyl benzoyl chloride at 0° C. The mixture was allowed stirred at 25° C. for overnight. After completion of the reaction, 16.8 g of AlCl₃ was added. To the mixture was added dropwise 19.5 g of 2-(o-tolyl)acetyl chloride at 0° C. The resulting mixture was allowed stirred at 25° C. for overnight. After cooling to 0° C., the solution of the reactor was slowly added to 200 mL of ice-water, and the organic layer was washed three times with water. The organic layer was dried over anhydrous MgSO₄, concentrated, and purified by column chromatography, giving 35 g (yield; 68%) of the 1-(9-(2-hydroxyethyl)-6-(thiophene-2-carbonyl)-9H-carbazol-3-yl)-2-(o-tolyl)ethanone.

¹H-NMR (δ, ppm, CDCl₃): 1.30 (t, 3H), 2.35 (s, 3H), 4.20 (s, 2H), 4.48 (m, 2H), 7.14-7.27 (m, 4H), 7.34 (m, 2H), 7.40 (m, 2H), 7.61-7.66 (m, 4H), 8.65 (s, 1H), 8.84 (s, 1H)

### 2 Step: Synthesis of (E)-1-(9-ethyl-6-(2-(trifluoromethyl)benzoyl)-9H-carbazol-3-yl)-2-(hydroxyimino)-2-(o-tolyl)ethanone

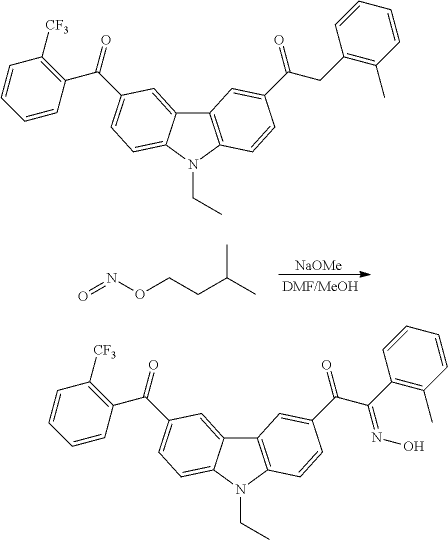

100 mL of dimethylformamide and 100 mL of methanol were placed in a reactor and 35.0 g of 1-(9-(2-hydroxyethyl)-6-(thiophene-2-carbonyl)-9H-carbazol-3-yl)-2-(o-tolyl)ethanone was dissolved therein. To the solution was added dropwise 16.42 g of isopentyl nitrite at 0° C. Following the dropwise addition, 6.5 g of sodium methoxide was added. The mixture was allowed to react with stirring at room temperature. After the reaction mixture was washed with 500 mL of dichloromethane and 300 mL of distilled water. After the resulting mixture was washed with water. The organic layer was dried over anhydrous MgSO₄ and concentrated, and purification by the column chromatography gave 27 g of the (E)-1-(9-ethyl-6-(2-(trifluoromethyl)benzoyl)-9H-carbazol-3-yl)-2-(hydroxyimino)-2-(o-tolyl)ethanone (yield; 73%).

¹H-NMR (δ, ppm, CDCl₃): 1.30 (t, 3H), 2.35 (s, 3H), 4.48 (m, 2H), 7.14-7.27 (m, 4H), 7.34 (m, 2H), 7.40 (m, 2H), 7.61-7.66 (m, 4H), 8.65 (s, 1H), 8.84 (s, 1H)

### 3 Step: Synthesis of (E)-2-(acetoxyimino)-1-(9-ethyl-6-(2-(trifluoromethyl)benzoyl)-9H-carbazol-3-yl)-2-(o-tolyl)ethanone

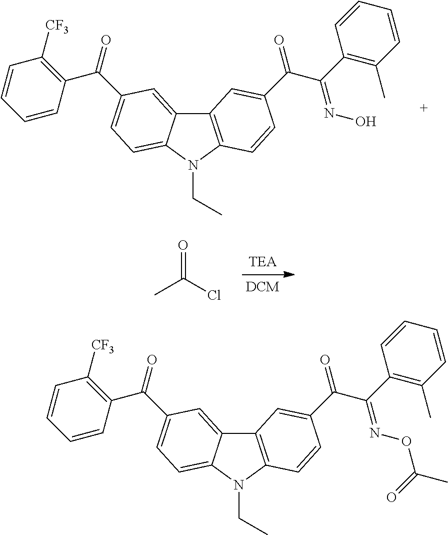

Under nitrogen atmosphere, 25.0 g of (E)-1-(9-ethyl-6-(2-(trifluoromethyl) benzoyl)-9H-carbazol-3-yl)-2-(hydroxyimino)-2-(o-tolyl)ethanone, 200 mL of dichloromethane and 6.5 g of triethylamine were added and a dilute solution of 4.3 g of acetyl chloride in 10 mL of dichloromethane was added dropwise thereto. The mixture was stirred for 5 h, 200 mL of water was added to the reaction solution to wash the organic layer twice and dried over anhydrous MgSO₄. The organic layer concentrated to obtain a solid. The solid was recrystallized from a mixture of ethyl acetate and hexane, followed by filtration to give 24 g of the (E)-2-(acetoxyimino)-1-(9-ethyl-6-(2-(trifluoromethyl)benzoyl)-9H-carbazol-3-yl)-2-(o-tolyl)ethanone as a pale yellow solid (yield; 89%).

¹H-NMR (δ, ppm, CDCl₃): 1.30 (t, 3H), 2.25 (s, 3H), 2.35 (s, 3H), 4.48 (m, 2H), 7.14-7.27 (m, 4H), 7.34 (m, 2H), 7.40 (m, 2H), 7.61-7.66 (m, 4H), 8.65 (s, 1H), 8.84 (s, 1H)

¹H NMR spectroscopic data for the compounds of Formulae 4-3 to 4-23 are shown in Table 1.

TABLE 1

| Formula | ¹H NMR Spectrum (CDCl₃, δ) |
|---|---|
| 4-3 | 2.21 (s, 3H), 3.09 (s, 3H), 4.57 (d, 4H), 7.27 (t, 1H), 7.53 (d, 1H), 7.58 (d, 1H), |
| | 7.60-7.73 (m, 3H), 7.75 (d, 2H), 7.98 (d, 1H), 8.11 (d, 1H), 8.23 (d, 2H), 8.50 (s, 1H), 8.72 (s, 1H) |
| 4-4 | 0.80-0.84 (t, 3H), 1.23-1.36 (m, 6H), 1.83-1.87 (m, 2H), 2.48 (s, 3H), 2.26 (s, 3H), |
| | 7.1-7.3 (m, 4H), 7.53 (d, 1H), 7.58 (d, 1H), 7.75 (d, 2H), 7.98 (d, 1H), 8.11 (d, 1H), 8.50 (s, 1H), |
| | 8.72 (s, 1H) |
| 4-5 | 2.21 (s, 3H), 2.25 (s, 3H), 2.48 (s, 3H), 4.57 (d, 4H), 7.27 (t, 1H), 7.53 (d, 1H), 7.58 (d, 1H), |
| | 7.75 (d, 2H), 7.98 (d, 1H), 8.11 (d, 1H), 8.50 (s, 1H), 8.72 (s, 1H) |
| 4-6 | 0.80-0.84 (t, 3H), 1.23-1.36 (m, 6H), 1.83-1.87 (m, 2H), 2.48 (s, 3H), 7.1-7.3 (m, 5H), |
| | 7.53 (d, 1H), 7.58 (d, 1H), 7.60-7.73 (m, 3H), 7.75 (d, 2H), 7.89 (d, 1H), 8.11 (d, 1H), 8.50 (s, |
| | 1H), 8.72 (s, 1H), 8.23 (d, 2H) |
| 4-7 | 1.09 (t, 3H), 2.53 (s, 3H), 3.41 (m, 2H), 3.85 (m, 2H), 4.54 (m, 2H), 7.1-7.3 (m, 5H), |
| | 7.53 (d, 1H), 7.58 (d, 1H), 7.60-7.70 (m, 3H), 7.75 (d, 2H), 7.98 (d, 1H), 8.11 (d, 1H), 8.50 (s, |
| | 1H), 8.72 (s, 1H), 8.23 (d, 2H) |
| 4-9 | 1.09 (t, 3H), 2.25 (s, 3H), 2.53 (s, 3H), 3.41 (m, 2H), 3.85 (m, 2H), 4.54 (m, 2H), 7.27 (t, |
| | 1H), 7.53 (d, 1H), 7.58 (d, 1H), 7.75 (d, 2H), 7.98 (d, 1H), 8.11 (d, 1H), 8.50 (s, 1H), |
| | 8.72 (s, 1H) |
| 4-10 | 1.09 (t, 3H), 2.21 (s, 3H), 3.41 (m, 2H), 3.85 (m, 2H), 4.54 (m, 2H), 7.27 (t, 1H), 7.53 (d, |
| | 1H), 7.58 (d, 1H), 7.60-7.73 (m, 3H), 7.75 (d, 2H), 7.98 (d, 1H), 8.11 (d, 1H), 8.50 (s, 1H), |
| | 8.72 (s, 1H), 8.23 (d, 2H) |
| 4-11 | 2.15 (s, 3H), 2.35 (s, 3H), 3.44 (t, 2H), 3.86 (t, 2H), 5.58 (dd, 1H), 6.03 (t, 1H), 6.27 (dd, |
| | 1H), 7.1-7.3 (m, 4H), 7.72 (m, 3H), 7.75 (t, 2H), 8.21-8.23 (d, 2H), 8.73 (d, 1H), 8.84 (d, |
| | 1H) |
| 4-12 | 2.25 (s, 3H), 2.53 (s, 3H), 3.44 (m, 2H), 3.86 (t, 2H), 5.58 (dd, 1H), 6.27 (dd, 1H), 6.03 (t, |
| | 1H), 7.27 (t, 1H), 7.53 (d, 1H), 7.58 (d, 1H), 7.75 (d, 2H), 7.98 (d, 1H), 8.11 (d, 1H), |
| | 8.50 (s, 1H), 8.72 (s, 1H) |
| 4-13 | 1.10 (t, 3H), 2.15 (s, 3H), 2.35 (s, 3H), 3.44 (m, 2H), 3.86 (m, 2H), 4.54 (m, 2H), |
| | 7.11-7.28 (m, 4H), 7.51-8.12 (m, 10H), 8.23 (s, 1H), 8.50 (s, 1H), 8.72 (s, 1H) |
| 4-14 | 2.15 (s, 3H), 2.35 (s, 3H), 3.44 (m, 2H), 3.86 (m, 2H), 5.58 (dd, 1H), 6.27 (dd, 1H), 6.30 (t, |
| | 1H), 7.13-7.33 (m, 4H), 7.50-8.08 (m, 10H), 8.21 (s, 1H), 8.53 (s, 1H), 8.70 (s, 1H) |
| 4-15 | 2.21 (s, 3H), 2.25 (s, 3H), 2.48 (s, 3H), 4.57 (m, 4H), 7.08-7.28 (m, 4H), 7.48-8.03 (m, |
| | 10H), 8.21 (s, 1H), 8.53 (s, 1H), 8.70 (s, 1H) |
| 4-16 | 1.49 (t, 3H), 2.26 (s, 3H), 2.48 (s, 3H), 4.43 (m, 2H), 7.11-7.30 (m, 4H), 7.72 (m, 3H), |
| | 7.75 (t, 2H), 8.21-8.23 (d, 2H), 8.73 (s, 1H), 8.84 (s, 1H) |
| 4-17 | 1.49 (t, 3H), 2.25 (s, 3H), 2.49 (s, 3H), 4.43 (m, 2H), 7.09-7.27 (m, 4H), 7.48-8.02 (m, |
| | 10H), 8.21 (s, 2H), 8.53 (s, 1H), 8.70 (s, 1H) |
| 4-18 | 0.81 (t, 3H), 1.21-1.35 (m, 6H), 1.83-1.87 (m, 2H), 2.26 (s, 3H), 2.48 (s, 3H), 7.08-7.28 (m, |
| | 4H), 7.50-8.01 (m, 10H), 8.21 (s, 1H), 8.50 (s, 1H), 8.68 (s, 1H) |
| 4-20 | 1.30 (t, 3H), 2.25 (s, 3H), 2.35 (s, 3H), 4.48 (m, 2H), 7.14-7.27 (m, 4H), 7.34 (m, 2H), |
| | 7.40 (m, 2H), 8.65 (s, 1H), 8.84 (s, 1H) |
| 4-21 | 1.30 (t, 3H), 2.25 (s, 3H), 2.35 (s, 3H), 4.26 (s, 2H), 4.48 (m, 2H), 5.73 (s, 1H), |
| | 7.14-7.27 (m, 4H), 7.34 (m, 2H), 7.40 (m, 2H), 7.61-7.66 (m, 4H), 8.65 (s, 1H), 8.84 (s, 1H) |
| 4-22 | 1.09 (t, 3H), 1.88 (t, 2H), 2.28 (s, 3H), 2.35 (s, 3H), 3.06 (t, 2H), 3.42 (m, 2H), 3.83 (t, 2H), |
| | 4.52 (t, 2H), 7.14-7.20 (m, 2H), 7.29-7.33 (m, 2H), 7.37-7.41 (m, 2H) 7.47 (d, 1H), 7.50 (d, |
| | 1H), 7.76 (d, 1H), 8.03 (d, 1H), 8.29 (s, 1H), 8.53 (s, 1H) |
| 4-23 | 1.10 (t, 3H), 1.89 (t, 2H), 2.27 (s, 3H), 3.06 (t, 2H), 3.42 (m, 2H), 3.83 (t, 2H), 4.52 (t, 2H), |
| | 7.33-7.39 (m, 5H), 7.28 (dd, 1H) 7.47 (d, 1H), 7.50 (d, 1H), 7.99 (d, 1H), 8.09 (d, 1H), |
| | 8.14 (d, 1H), 8.59 (s, 1H), 8.83 (s, 1H) |

### EXAMPLE 2

### Synthesis of the Compound of Formula 5-1

### 1 Step: Synthesis of 1-(9-(2-ethoxyethyl)-6-(thiophene-2-carbonyl)-9H-carbazol-3-yl)ethanone

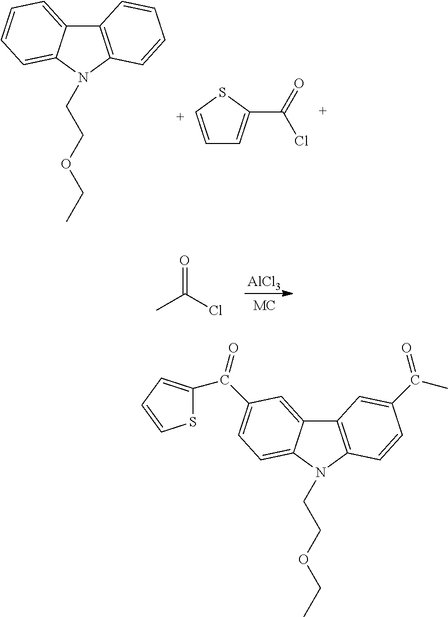

23.9 g of the 9-(2-ethoxyethyl)-9H-carbazole as a reactant and 100 mL of dichloromethane were placed in a 250 mL round-bottomed flask, 15.8 g of aluminum chloride was added at 0° C., and a dilute solution of 17.3 g of thiophene carbonyl chloride in 16 mL of dichlomethane was added dropwise thereto. Following stirring at 25° C. for 5 h, 15.8 g of aluminum chloride was added and 9.5 g of acetyl chloride was added dropwise. The mixture was allowed to react at 25° C. for 5 h. After completion of the reaction, the resulting mixture was washed with water, dried over anhydrous MgSO₄, and filtered. Concentration of the organic layer gave 31.3 g (yield; 80%) of the 1-(9-(2-ethoxyethyl)-6-(thiophene-2-carbonyl)-9H-carbazol-3-yl)ethanone as a solid.

¹H NMR (δ, ppm, CDCl₃): 1.10 (t, 3H), 2.50 (s, 3H), 3.50 (q, 2H), 3.85 (t, 2H), 4.45 (t, 2H), 7.27 (t, 1H), 7.56 (d, 1H), 7.74 (d, 1H), 7.84 (d, 1H), 8.0 (d, 1H), 8.14 (d, 1H), 8.60 (s, 1H), 8.90 (s, 1H)

### 2 Step: Synthesis of (E)-(9-(2-ethoxyethyl)-6-(1-(hydroxyimino)ethyl)-9H-carbazol-3-yl)(thiophen-2-yl)methanone

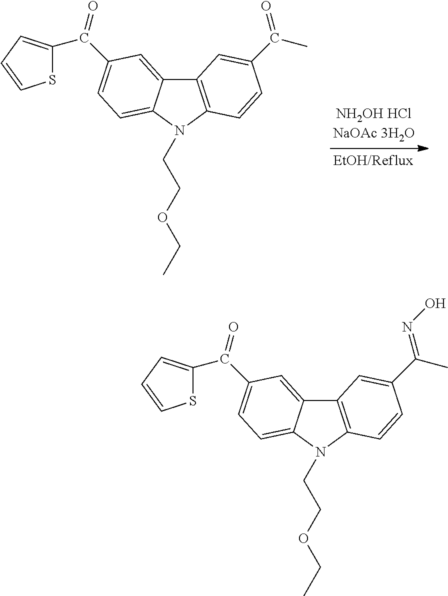 [SMILES: CCOCCn1c2ccc(C(C)=O)cc2c2cc(C(=O)c3cccs3)ccc21 ; CCOCCn1c2ccc(C(=O)c3cccs3)cc2c2cc(/C(C)=N/O)ccc21]

3.9 g of hydroxylamine hydrochloride and 5.4 g of sodium acetate were dissolved in 50 mL of distilled water in a 250 mL round-bottomed flask, and a solution of 19.8 g (0.05 mol) of the 1-(9-(2-ethoxyethyl)-6-(thiophene-2-carbonyl)-9H-carbazol-3-yl)ethanone in 150 mL of ethanol was added thereto. The reaction mixture was refluxed for 7 h. The reaction solution was added to ice-water to form a precipitate. The precipitate was collected by filtration and washed with distilled water to obtain a white solid. The solid was vacuum dried gave 18.7 g of the (E)-(9-(2-ethoxyethyl)-6-(1-(hydroxyimino)ethyl)-9H-carbazol-3-yl)(thiophen-2-yl)methanone (yield: 92%).

¹H NMR (δ, ppm, DMSO-d₆): 1.11 (t, 3H), 2.0 (s, 1H), 2.85 (s, 3H), 3.50 (m, 2H), 3.85 (t, 2H), 4.45 (t, 2H), 7.27 (t, 1H), 7.56 (d, 1H), 7.74 (d, 1H), 8.0 (t, 1H), 8.14 (m, 1H), 8.60 (s, 1H), 8.94 (s, 1H)

### 3 Step: Synthesis of (E)-1-(((1-(9-(2-ethoxyethyl)-6-(thiophene-2-carbonyl)-9H-carbazol-3-yl)ethylidene)amino)oxy)ethanone

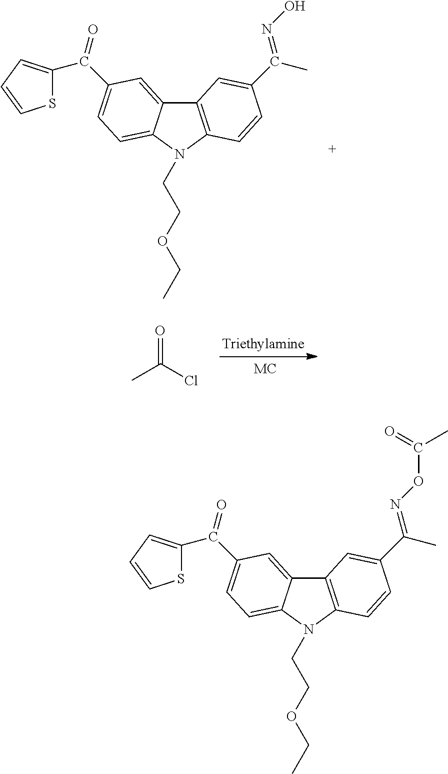

19 g of the (E)-(9-(2-ethoxyethyl)-6-(1-(hydroxyimino)ethyl)-9H-carbazol-3-yl) (thiophen-2-yl)methanone and 100 mL of dichloromethane were placed in a 250 mL round-bottomed flask, 4.6 g of triethylamine was added and 3.38 g (0.041 mol, 1.1 eq.) of acetyl chloride was added dropwise thereto at 0° C. Following stirring at 25° C. for 5 h, the reaction mixture was added to 50 mL of water. The organic layer was washed with water, dried over anhydrous MgSO₄, filtered, and concentrated, giving 16.8 g (yield; 80%) of the (E)-1-(((1-(9-(2-ethoxyethyl)-6-(thiophene-2-carbonyl)-9H-carbazol-3-yl)ethylidene)amino)oxy)ethanone as a solid.

₁H NMR (δ, ppm, CDC1₃): 1.10 (t, 3H), 2.28 (s, 3H), 2.85 (s, 3H), 3.50 (q, 2H), 3.85 (t, 2H), 4.45 (t, 2H), 7.27 (t, 1H), 7.56 (d, 1H), 7.74 (d, 1H), 7.97-8.00 (t, 2H), 8.14-8.18 (m, 2H), 8.60 (s, 1H), 8.94 (s, 1H)

### Synthesis of the Compound of Formula 5-2

### 1 Step: Synthesis of 1-(9-(2-ethoxyethyl)-6-(thiophene-2-carbonyl)-9H-carbazol-3-yl)ethanone

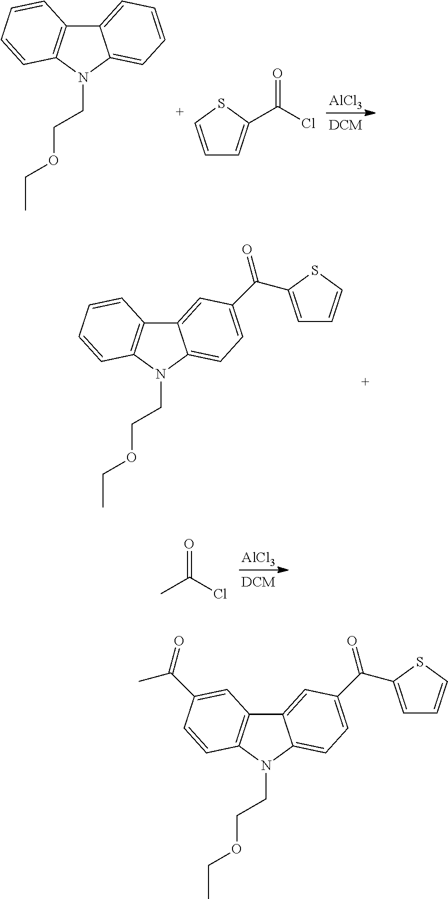

400 g of the 9-(2-ethoxyethyl)-9H-carbazole as a reactant and 4000 mL of methylene chloride were placed in a 10 L round-bottomed flask, 230.2 of aluminum chloride was added thereto at 0° C., and 280.1 g of thiophene carbonyl chloride in 250 mL of dichloromethane was added dropwise thereto. Following stirring at 25° C. for 5 h, 280.1 g of aluminum chloride was added at 0° C. and 140.3 g of acetyl chloride was added dropwise. Following stirring at 25° C. for 5 h, the reaction mixture was slowly added to ice-water. The organic layer was washed with water, dried over anhydrous MgSO₄, and filtered. Concentration of the organic layer gave 558 g (yield; 86%) of the 1-(9-(2-ethoxyethyl)-6-(thiophene-2-carbonyl)-9H-carbazol-3-yl)ethanone as a solid.

¹H NMR (δ, ppm, CDCl₃): 1.09 (t, 3H), 2.74 (s, 3H), 3.43 (q, 2H), 3.86 (t, 2H), 4.57 (t, 2H), 7.24 (dd, 1H), 7.56 (d, 1H), 7.60 (d, 1H), 7.76 (m, 1H), 8.13 (dd, 1H), 8.18 (dd, 1H), 8.75 (s, 1H), 8.77 (s, 1H)

### 2 Step: Synthesis of (E)-(9-(2-ethoxyethyl)-6-(1-(hydroxyimino)ethyl)-9H-carbazol-3-yl)(thiophen-2-yl)methanone

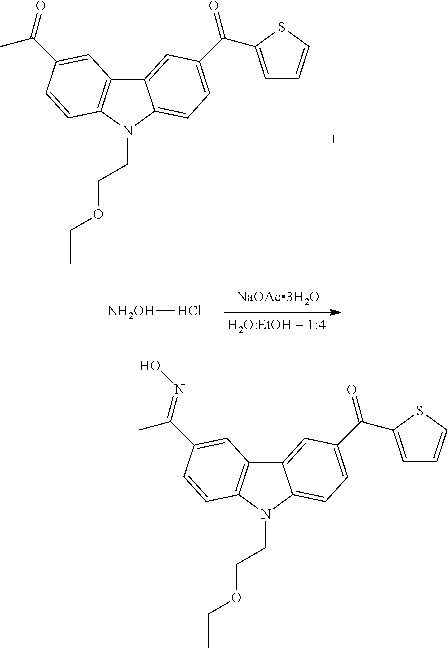

109.5 g of hydroxylamine hydrochloride and 210.7 g of sodium acetate were dissolved in 700 mL of distilled water in a 5000 mL round-bottomed flask, and a solution of 550 g of the 1-(9-(2-ethoxyethyl)-6-(thiophene-2-carbonyl)-9H-carbazol-3-yl)ethanone as a solid in 2640 mL of ethanol was added thereto. The solution was refluxed for 7 h. The reaction solution was added to ice-water to form a precipitate. The precipitate was collected by filtration and washed with distilled water to obtain a white solid. The solid was vacuum dried gave 350 g of the (E)-(9-(2-ethoxyethyl)-6-(1-(hydroxyimino)ethyl)-9H-carbazol-3-yl)(thiophen-2-yemethanone as a solid (yield; 62%).

¹H NMR (δ, ppm, CDCl₃): 1.10 (t, 3H), 2.43 (s, 3H), 3.43 (t, 2H), 3.85 (t, 2H), 4.54 (t, 2H), 7.22 (t, 1H), 7.51 (d, 1H), 7.56 (d, 1H), 7.74 (t, 1H), 7.87 (m, 1H), 8.38 (s, 1H), 8.71 (s, 1H)

### 3 Step: Synthesis of (E)-1-(((1-(9-(2-ethoxyethyl)-6-(thiophene-2-carbonyl)-9H-carbazol-3-yl)ethylidene)amino)oxy)ethanone

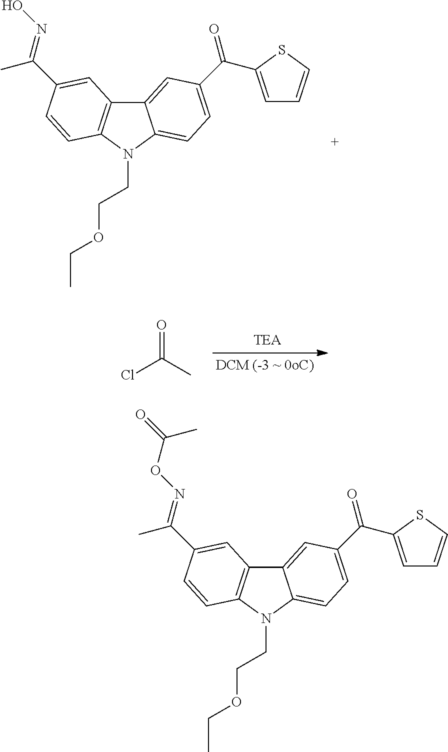

350 g of the (E)-(9-(2-ethoxyethyl)-6-(1-(hydroxyimino)ethyl)-9H-carbazol-3-yl)(thiophen-2-yl)methanone and 2 L of methylene chloride were placed in a 5 L round-bottomed flask, 108.3 g of triethylamine was added and 77.3 g of acetyl chloride was added thereto at 0° C. Following stirring at 25° C. for 5 h, the reaction mixture was added to 2 L of water. The organic layer was washed with water, dried over anhydrous MgSO₄, and filtered. Concentration of the organic layer gave 280 g (yield; 73%) of the (E)-1-(((1-(9-(2-ethoxyethyl)-6-(thiophene-2-carbonyl)-9H-carbazol-3-yl)ethylidene)amino)oxy)ethanone as a solid.

¹H NMR (δ, ppm, CDCl₃): 1.09 (t, 3H), 2.30 (s, 3H), 2.53 (s, 3H), 3.41 (q, 2H), 3.85 (t, 2H), 4.54 (t, 2H), 7.27 (t, 1H), 7.53 (d, 1H), 7.58 (d, 1H), 7.75 (d, 2H), 7.98 (d, 1H), 8.11 (d, 1H), 8.50 (s, 1H), 8.72 (s, 1H)

### Synthesis of the Compound of Formula 5-16

### 1 Step: Synthesis of 1-(6-(2-naphthoyl)-6-(ethoxyethyl)-9H-carbazol-3-yl)ethanone

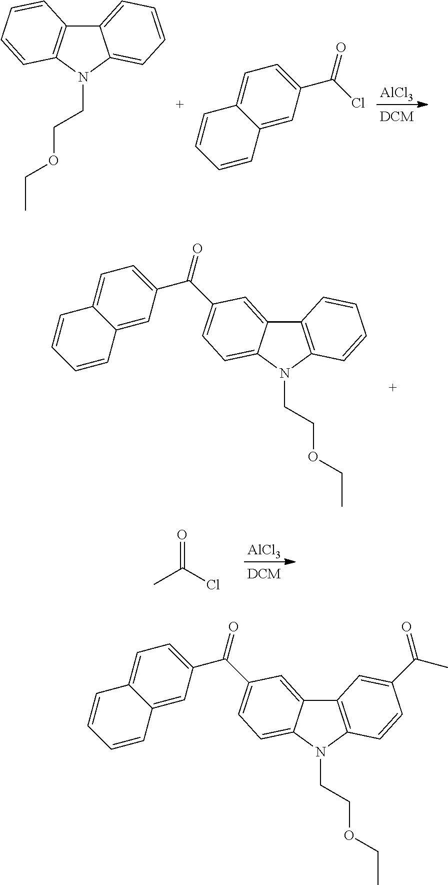

400 g of the 9-(2-ethoxyethyl)-9H-carbazole as a reactant and 4000 mL of dichloromethane were placed in a 10 L round-bottomed flask, 245.3 g of aluminum chloride was added at 0° C., and 320.5 g of 1-naphthoyl chloride was added dropwise thereto. Following stirring at 25° C. for 5 h, the reaction was stopped. 245.3 g of aluminum chloride was added at 0° C. and 142.1 g of acetyl chloride was added dropwise. Following stirring at 25° C. for 5 h, the reaction mixture was slowly added to 4 L of ice-water. The organic layer was washed with water, dried over anhydrous MgSO₄, and filtered. Concentration of the organic layer gave 582 g (yield; 80%) of the 1-(6-(2-naphthoyl)-6-(ethoxyethyl)-9H-carbazol-3-yl)ethanone as a solid.

¹H NMR (δ, ppm, CDCl₃): 1.09 (t, 3H), 2.74 (s, 3H), 3.43 (q, 2H), 3.86 (t, 2H), 4.57 (t, 2H), 7.13-7.33 (m, 4H), 7.50-8.08 (m, 6H), 8.22 (s, 1H), 8.50 (s, 1H), 8.86 (s, 1H)

### 2 Step: Synthesis of (E)-(9-(2-ethoxyethyl)-6-(1-(hydroxyimino)ethyl)-9H-carbazol-3-yl)(naphthalene-2-yl)methanone

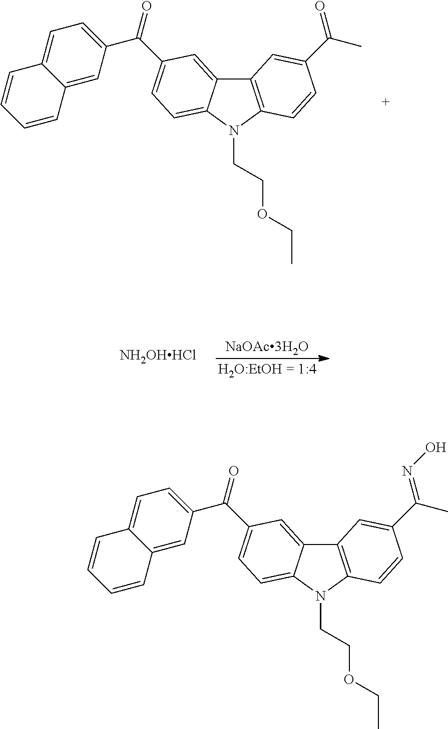 [SMILES: CCOCCn1c2ccc(C(C)=O)cc2c2cc(C(=O)c3ccc4ccccc4c3)ccc21 ; "NH2OH·HCl" ; CCOCCn1c2ccc(C(=O)c3ccc4ccccc4c3)cc2c2cc(/C(C)=N/O)ccc21]

110.1 g of hydroxylamine hydrochloride and 219. 6 g of sodium acetate were dissolved in 600 mL of distilled water in a 5000 mL round-bottomed flask, and a solution of 609.7 g of the 1-(6-(2-naphthoyl)-6-(ethoxyethyl)-9H-carbazol-3-yl)ethanone in 2 L of ethanol was added thereto. The reaction mixture was refluxed for 7 h. After the reaction mixture was added to ice-water to form a precipitate. The precipitate was collected by filtration and washed with distilled water to obtain a white solid. The solid was dried over vacuum gave 391 g (yield; 62%) of the (E)-(9-(2-ethoxyethyl)-6-(1-(hydroxyimino)ethyl)-9H-carbazol-3-yl)(naphthalene-2-yl)methanone as a solid.

¹H NMR (δ, ppm, CDCl₃): 1.09 (t, 3H), 2.43 (s, 3H), 3.43 (q, 2H), 3.85 (t, 2H), 4.54 (t, 2H), 7.12-7.32 (m, 4H), 7.49-8.06 (m, 6H), 8.23 (s, 1H), 8.52 (s, 1H), 8.87 (s, 1H)

### 3 Step: Synthesis of (E)-1-(((1-(9-(2-naphthoyl)-6-(2-ethoxyethyl)-9H-carbazol-3-yl)ethylidene)amino)oxy)ethanone

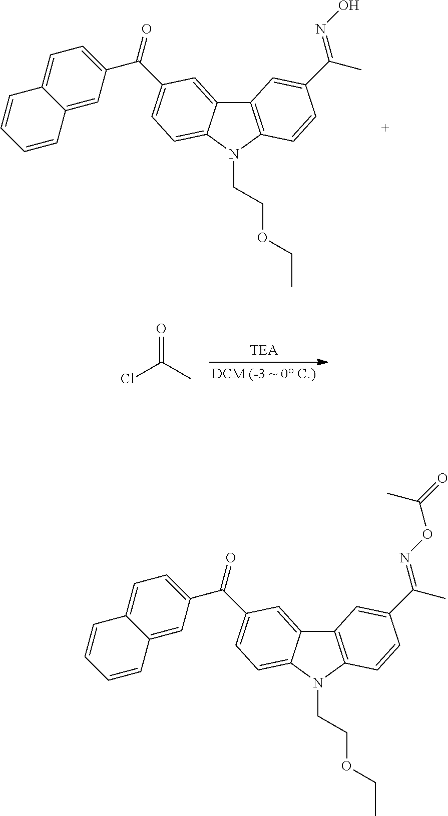

388 g of the (E)-(9-(2-ethoxyethyl)-6-(1-(hydroxyimino)ethyl)-9H-carbazol-3-yl)(naphthalene-2-yl)methanone and 2 L of dichloromethane were placed in a 5 L round-bottomed flask, 110.2 g of triethylamine was added at 0° C., and 80.3 g of acetyl chloride was added thereto. Following stirring at 25° C. for 5 h, the reaction mixture was added to 2 L of water. The organic layer was washed with water, dried over anhydrous MgSO₄, and filtered. Concentration of the organic layer gave 318 g (yield; 75%) of the (E)-1-(((1-(9-(2-naphthoyl)-6-(2-ethoxyethyl)-9H-carbazol-3-yl)ethylidene)amino)-oxy)ethanone as a solid.

¹H NMR (δ, ppm, CDCl₃): 1.10 (t, 3H), 2.14 (s, 3H), 2.35 (s, 3H), 3.44 (m, 2H), 3.86 (t, 2H), 4.54 (t, 2H), 7.13-7.33 (m, 4H), 7.50-8.08 (m, 6H), 8.21 (s, 1H), 8.53 (s, 1H), 8.70 (s, 1H)

### Synthesis of the Compound of Formula 5-19

### 1 Step: Synthesis of 4-chloro-1-(9-(2-ethoxyethyl)-6-(2-methylbenzoyl)-9H-carbazol-3-yl)butan-1-one

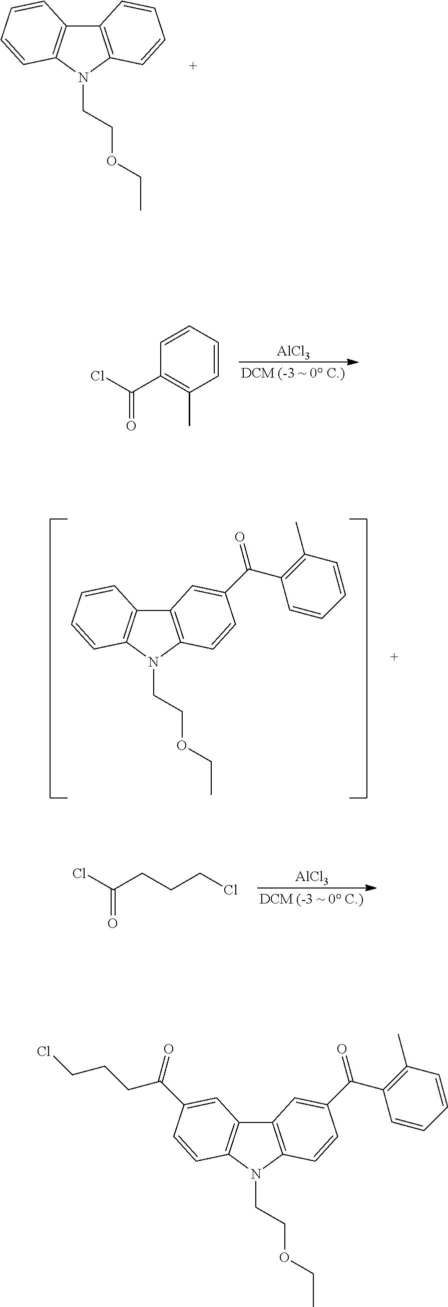

50 g of the 9-(2-ethoxyethyl)-9H-carbazole as a reactant and 400 mL of methylene chloride were placed in a 1000 mL round-bottomed flask, 31.5 g of aluminum chloride was added at 0° C., and a dilute solution of 37.3 g of o-toluoyl chloride in 60 mL of dichloromethane was added dropwise thereto. Following stirring at 25° C. for 5 h, the reaction was stopped. 33.2 g of aluminum chloride was added at 0° C. and 36.5 g of 4-chlorobutanoyl chloride was added dropwise. Following stirring at 25° C. for 5 h, the reaction mixture was slowly added to a mixture 600 mL of ice-water. The organic layer was washed with water, dried over anhydrous MgSO₄, and filtered. Concentration of the organic layer gave 80 g (yield; 82%) of the 4-chloro-1-(9-(2-ethoxyethyl)-6-(2-methylbenzoyl)-9H-carbazol-3-yl)butan-1-one as a solid.

¹H NMR (δ, ppm, CDCl₃): 1.09 (t, 3H), 2.74 (s, 3H), 3.43 (q, 2H), 3.86 (t, 2H), 4.57 (t, 2H), 7.24 (dd, 1H), 7.56 (d, 1H), 7.60 (d, 1H), 7.76 (m, 1H), 8.13 (dd, 1H), 8.18 (dd, 1H), 8.75 (s, 1H), 8.77 (s, 1H)

### 2 Step: Synthesis of 4-((4-chlorophenyl)thio)-1-(9-(2-ethoxyethyl)-6-(2-methylbenzoyl)-9H-carbazol-3-yl)butan-1-one

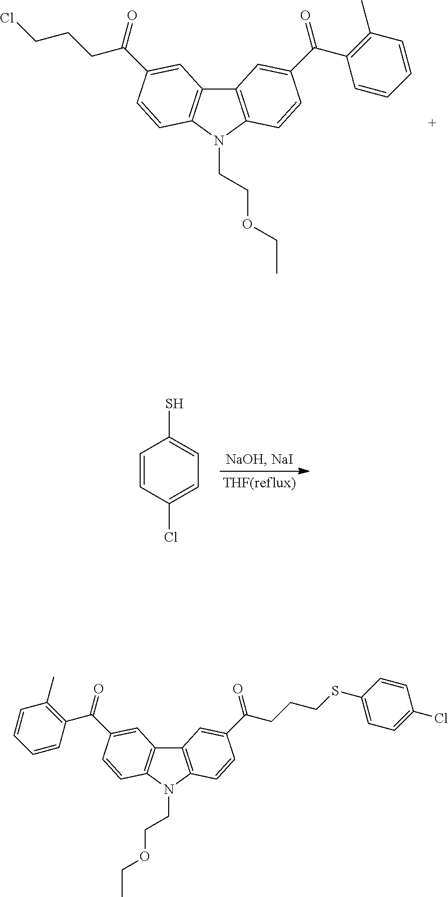 [SMILES: CCOCCn1c2ccc(C(=O)CCCCl)cc2c2cc(C(=O)c3ccccc3C)ccc21 ; Sc1ccc(Cl)cc1 ; CCOCCn1c2ccc(C(=O)CCCSc3ccc(Cl)cc3)cc2c2cc(C(=O)c3ccccc3C)ccc21]

80 g of the 4-chloro-1-(9-(2-ethoxyethyl)-6-(2-methylbenzoyl)-9H-carbazol-3-yl)butan-1-one and 30.0 g of sodium iodide were dissolved in 300 mL of acetone. The reaction mixture was refluxed for 12 h. After cooling to 25° C., 300 ml of tetrahydrofuran and 8.0 g of sodium hydroxide was sequentially added and a solution of 30.3 g of chlorobenzenethiol in 50 ml of tetrahydrofuran was added. The mixture was allowed to react under refluxing for 3 h. After the reaction mixture was evaporated to remove solvents, dissolved in dichloromethane, washed with distilled water, dried over anhydrous MgSO₄, and filtered. The organic layer was concentrated and purified by column chromatography, giving 82 g of the 4(4-chlorophenyl)thio)-1-(9-(2-ethoxyethyl)-6-(2-methylbenzoyl)-9H-carbazol-3-yl)butan-1-one as a solid (yield; 85%).

¹H NMR (δ, ppm, CDCl₃): 1.07 (t, 3H), 2.14 (m, 2H), 2.36 (s, 3H), 3.06 (t, 2H), 3.25 (t, 2H), 3.41 (t, 2H), 3.84 (t, 2H), 4.54 (t, 2H), 7.22-7.44 (m, 8H), 7.53 (dd, 2H), 8.09 (d, 1H), 8.13 (d, 1H), 8.54 (s, 1H), 8.70 (s, 1H)

### 3 Step: Synthesis of (6-(4-((4-chlorophenyl)thio)-1-(hydroxyimino)butyl)-9-(2-ethoxyethyl)-9H-carbazol-3-yl)(o-tolyl)methanone

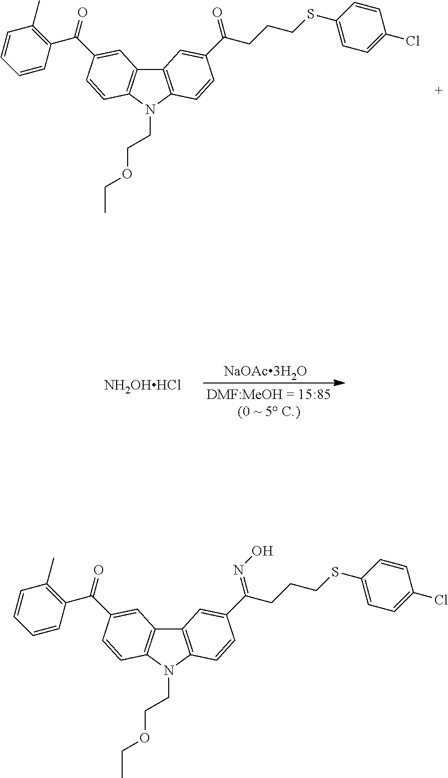 [SMILES: CCOCCn1c2ccc(C(=O)CCCSc3ccc(Cl)cc3)cc2c2cc(C(=O)c3ccccc3C)ccc21 ; "NH2OH·HCl" ; CCOCCn1c2ccc(C(=O)c3ccccc3C)cc2c2cc(/C(CCCSc3ccc(Cl)cc3)=N/O)ccc21]

0.75 g of hydroxylamine hydrochloride and 1.5 g of sodium acetate were dissolved in 10 mL of distilled water in a 100 mL round-bottomed flask, and a solution of 5 g of the 4-((4-chlorophenyl)thio)-1-(9-(2-ethoxyethyl)-6-(2-methylbenzoyl)-9H-carbazol-3-yl)butan-1-one in 40 mL of ethanol was added thereto. The reaction mixture was refluxed for 7 h. The reaction mixture was added to ice-water to form a precipitate. The precipitate was collected by filtration and washed with distilled water to obtain a white solid. The solid was dissolved in dichloromethane, dried over anhydrous MgSO₄, and filtered. The organic layer was concentrated and purified by column chromatography, giving 5 g of the (6-(4-((4-chlorophenyl)thio)-1-(hydroxyimino)butyl)-9-(2-ethoxyethyl)-9H-carbazol-3-yl)(o-tolyl)methanone as a solid (yield; 88%).

¹H NMR (δ, ppm, CDCl₃): 1.09 (t, 3H), 1.94 (t, 2H), 2.35 (s, 3H), 2.97 (t, 2H), 3.06 (t, 2H), 3.42 (m, 2H), 3.83 (t, 2H), 4.52 (t, 2H), 7.14-7.20 (m, 2H), 7.29-7.33 (m, 2H), 7.37-7.41 (m, 2H), 7.47 (d, 1H), 7.50 (d, 1H), 7.76 (d, 1H), 8.03 (d, 1H), 8.29 (s, 1H), 8.53 (s, 1H)

### 4 Step: Synthesis of 1-(((4-((4-chlorophenyl)thio)-1-(9-(2-ethoxyethyl)-6-(2-methylbenzoyl)-9H-carbazol-3-yl)butylidene)amino)oxy)ethanone

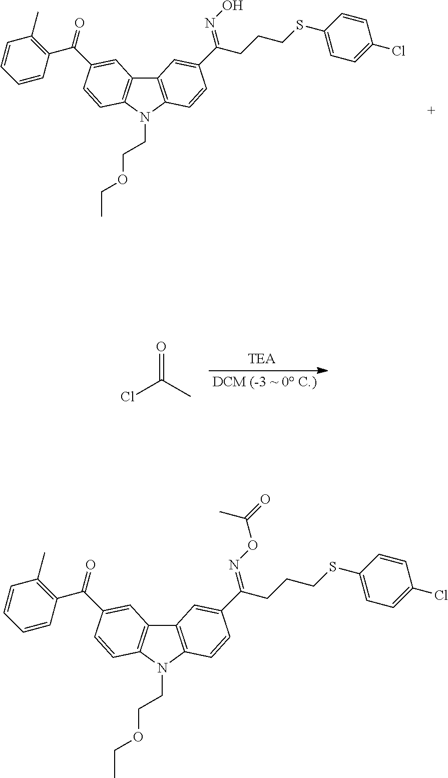

3 g of the (6-(4-((4-chlorophenyl)thio)-1-(hydroxyimino)butyl)-9-(2-ethoxyethyl)-9H-carbazol-3-yl)(o-tolyl)methanone and 30 mL of dichloromethane were placed in a 250 mL round-bottomed flask, 0.7 g of triethylamine was added thereto at 0° C., and 0.6 g of acetyl chloride was added dropwise thereto. Following stirring at 25° C. for 5 h, the reaction mixture was added to 50 mL of water. The organic layer was washed with water, dried over anhydrous MgSO₄, and filtered. Concentration of the organic layer gave 2.0 g (yield; 62%) of the 1-(((4-((4-chlorophenyl)thio)-1-(9-(2-ethoxyethyl)-6-(2-methylbenzoyl)-9H-carbazol-3-yl)butylidene)amino)oxy)ethanone as a solid.

¹H NMR (δ, ppm, CDCl₃): 1.09 (t, 3H), 1.94 (t, 2H), 2.28 (s, 3H), 2.35 (s, 3H), 2.97 (t, 2H), 3.06 (t, 2H), 3.42 (m, 2H), 3.83 (t, 2H), 4.52 (t, 2H), 7.14-7.20 (m, 2H), 7.29-7.33 (m, 2H), 7.37-7.41 (m, 2H) 7.47 (d, 1H), 7.50 (d, 1H), 7.76 (d, 1H), 8.03 (d, 1H), 8.29 (s, 1H), 8.53 (s, 1H)

### Synthesis of the Compound of Formula 5-20

### 1 Step: Synthesis of 1-(9-(2-ethoxyethyl)-6-(thiophene-2-carbonyl)-9H-carbazol-3-yl)-2-(o-tolyl)ethanone

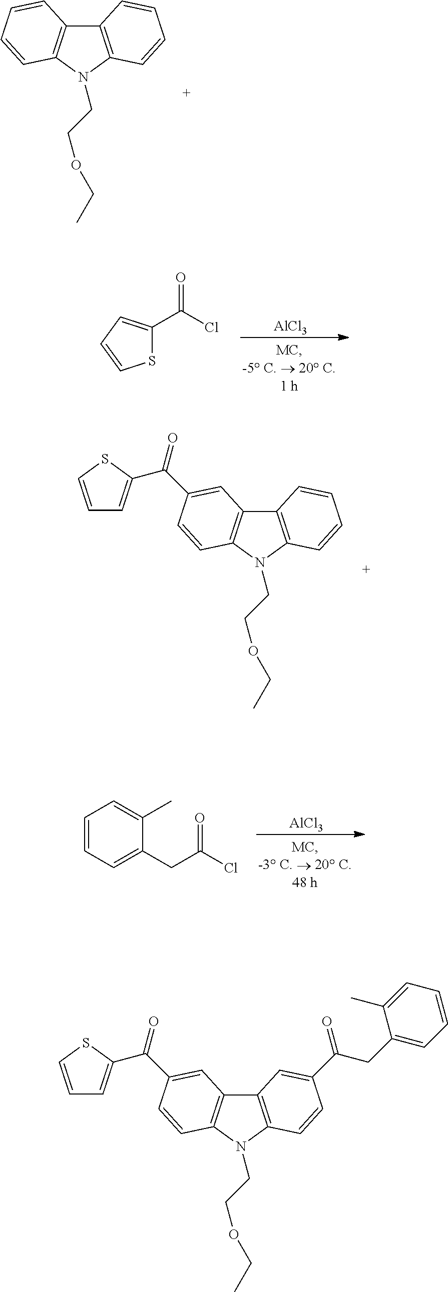

50.0 g of the 9-(2-ethoxyethyl)-9H-carbazole as a reactant was dissolved in 500 ml of dry dichloromethane. After the reaction mixture was cooled to 0° C., 30.2 g of AlCl₃ was added thereto. To the resulting mixture was added dropwise a mixture of 34.2 g of thiophene carbonyl chloride and 30 ml of dry dichloromethane at 0° C. The reaction mixture was stirred at 25° C. for 1 h. After the reaction mixture was cooled to 0° C., 30.2 g of AlCl₃ was added thereto. To the mixture were added dropwise 40.2 g of 4-chlorobutanoyl chloride and 30 ml of dichloromethane at 0° C. The reaction mixture was allowed to proceed at a temperature of 25° C. for 24 h. After completion of the reaction, the organic layer was washed with water. Thereafter, the organic layer was dried over anhydrous MgSO₄ and concentrated to obtain a solid. The solid was vacuum dried, giving 70.58 g (yield; 72%) of the 1-(9-(2-ethoxyethyl)-6-(thiophene-2-carbonyl)-9H-carbazol-3-yl)-2-(o-tolyl)ethanone.

¹H-NMR (δ, ppm, CDCl₃): 1.07 (t, 3H), 2.31 (s, 3H), 3.41 (q, 2H), 3.83 (t, 2H), 4.43 (s, 2H), 4.52 (t, 2H), 7.20 (m, 5H), 7.53 (m, 2H), 7.73 (m, 2H), 8.09 (d, 1H), 8.21 (d, 1H), 8.71 (s, 1H), 8.82 (s, 1H).

### 2 Step: Synthesis of (E)-(9-(2-ethoxyethyl)-6-(1-(hydroxyimino)-2-(o-tolyl)ethyl)-9H-carbazol-3-yl)(thiophen-2-yl)methanone

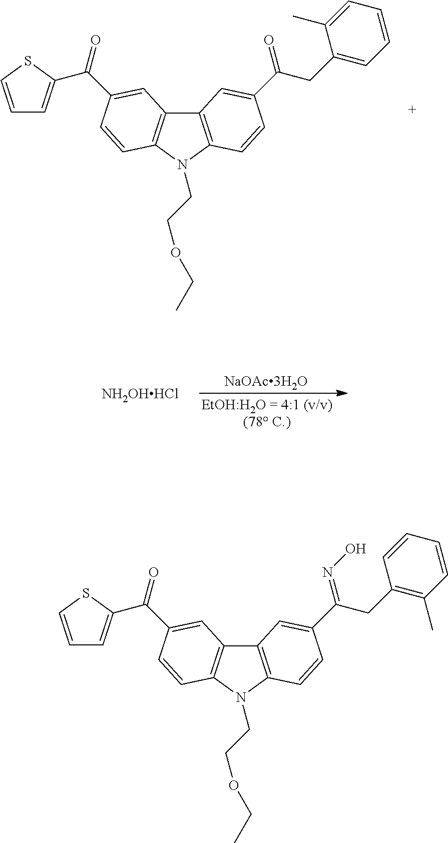 [SMILES: CCOCCn1c2ccc(C(=O)Cc3ccccc3C)cc2c2cc(C(=O)c3cccs3)ccc21 ; "NH2OH·HCl" ; CCOCCn1c2ccc(C(=O)c3cccs3)cc2c2cc(/C(Cc3ccccc3C)=N/O)ccc21]

70.6 g of the 1-(9-(2-ethoxyethyl)-6-(thiophene-2-carbonyl)-9H-carbazol-3-yl)-2-(o-tolyl)ethanone and 800 ml of ethanol were placed in a reactor, and 13.5 g of hydroxylamine hydrochloride, 24.6 g of sodium acetate in 300 ml of distilled water was added thereto. The reaction mixture was stirred at 70° C. After 15 h, the reaction mixture was concentrated to remove the ethanol. The concentrate was dissolved in 500 ml of dichloromethane and washed twice with 500 ml of distilled water. After the solution was dried over anhydrous MgSO₄, and concentrated to obtain a solid. The solid was vacuum dried, giving 45.3 g (yield; 62%) of the (E)-(9-(2-ethoxyethyl)-6-(1-(hydroxyimino)-2-(o-tolyl)ethyl)-9H-carbazol-3-yl)(thiophen-2-yl)methanone.

¹H-NMR (δ, ppm, CDCl₃):1.08 (t, 3H), 1.62 (s, 1H), 2.43 (s, 3H), 3.41 (q, 2H), 3.81 (t, 2H), 4.24 (s, 2H), 4.49 (t, 2H), 7.10 (m, 5H), 7.51 (m, 2H), 7.71 (m, 2H), 7.77 (d, 1H), 8.05 (d, 1H), 8.34 (s, 1H), 8.61 (s, 1H).

### 3 Step: Synthesis of (E)-1-(((1-(9-(2-ethoxyethyl)-6-(thiophene-2-carbonyl)-9H-carbazol-3-yl)-2-(o-tolyl)ethylidene)amino)oxy)ethanone

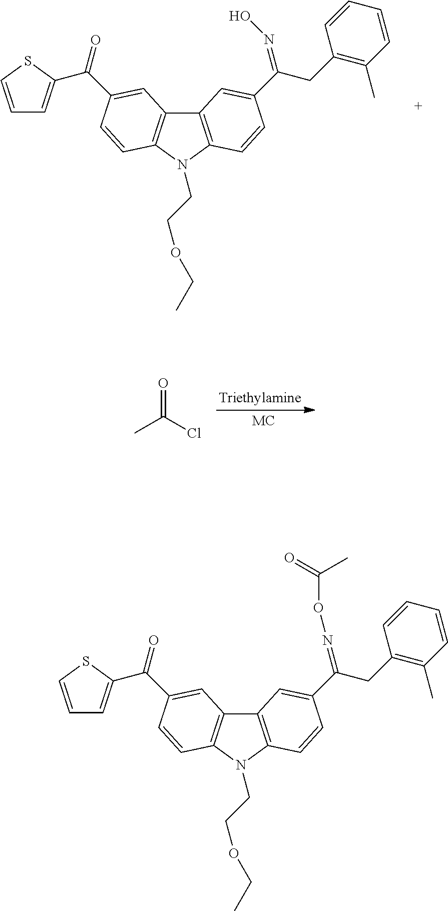

45.3 g of the (E)-(9-(2-ethoxyethyl)-6-(1-(hydroxyimino)-2-(o-tolyl)ethyl)-9H-carbazol-3-yl)(thiophen-2-yl)methanone and 450 ml of dichloromethane were placed in a reactor, 11.5 g of triethylamine was added at 0° C., and 8.5 g of acetyl chloride was added dropwise thereto. The reaction mixture was stirred for 12 h. The reaction mixture was washed three times with 200 ml of distilled water. The organic layer was distilled to remove the solvents. The residue was triturated with ethylacetate/n-Hexane. The solid was collected by filtration and dried. The solid was vacuum dried, giving 37 g of the (E)-1-(((1-(9-(2-ethoxyethyl)-6-(thiophene-2-carbonyl)-9H-carbazol-3-yl)-2-(o-tol-yl) ethylidene)amino)oxy)ethanone.

¹H-NMR (δ, ppm, CDCl₃): 1.07 (t, 3H), 2.18 (s, 3H), 2.43 (s, 3H), 3.40 (q, 2H), 3.81 (t, 2H), 4.29 (s, 2H), 4.51 (t, 2H), 7.12 (m, 5H), 7.46 (d, 1H), 7.53 (d, 1H), 7.73 (m, 2H), 7.87 (d, 1H), 8.07 (d, 1H), 8.49 (s, 1H), 8.65 (s, 1H).

### Synthesis of the Compound of Formula 5-21

### 1 Step: Synthesis of 1-(9-(2-ethoxyethyl)-6-(2-methylbenzoyl)-9H-carbazol-3-yl)ethanone

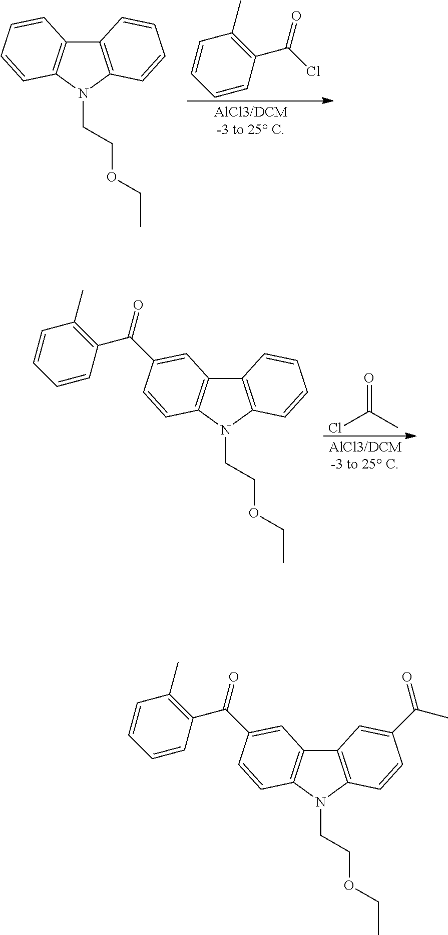

30.0 g of 9-(2-ethoxyethyl)-9H-carbazole was dissolved in 210 ml of dichloromethane. The solution was cooled to 0° C., and 16.5 g of AlCl₃ was added thereto. To the mixture was slowly added dropwise 18.3 g of o-toluoyl chloride at 0° C. The reaction mixture was stirred at 25° C. for 4 h. After the reaction mixture was cooled to 0° C., 16.5 g of AlCl₃ was added thereto. To the mixture were added dropwise 9.6 g of acetyl chloride at 0° C. The reaction mixture was stirred overnight. After finished reaction, the reaction mixture was slowly added to ice-water and washed with water. The organic layer was dried over anhydrous MgSO₄ and concentrated, giving 48 g of the 1-(9-(2-ethoxyethyl)-6-(2-methylbenzoyl)-9H-carbazol-3-yl)ethanone.

¹H-NMR (δ, ppm, CDCl₃): 1.06-1.08 (t, 3H), 2.3 (t, 3H), 2.7 (t, 3H), 3.4 (d, 2H), 3.8 (d, 2H), 4.5 (d, 2H), 7.3-7.4 (s, 4H), 7.5 (s, 2H), 8.0 (s, 1H), 8.1 (s, 1H), 8.5 (s, 1H), 8.6 (s, 1H)

### 2 Step: Synthesis of (E)-(9-(2-ethoxyethyl)-6-(1-(hydroxyimino)ethyl)-9H-carbazol-3-yl)(o-tolyl)methanone

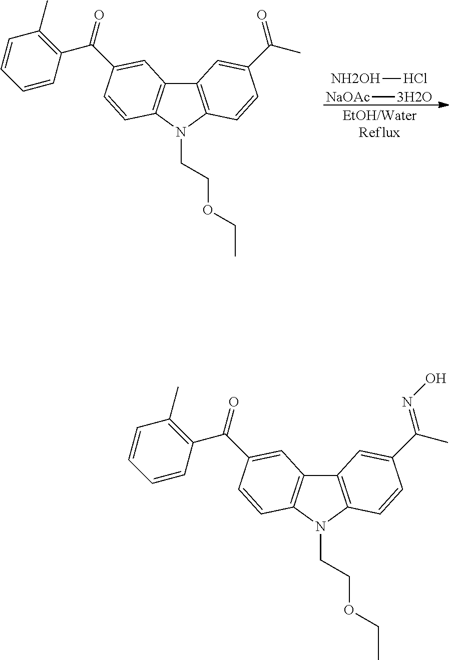

40.0 g of the 1-(9-(2-ethoxyethyl)-6-(2-methylbenzoyl)-9H-carbazol-3-yl)ethanone, ethyl alcohol and water were added to dissolve the compound. To the reaction mixture was added 9.3 g of hydroxylamine chloride and 15.4 g of sodium acetate trihydrate. The reaction mixture was refluxed for 5 h. After the reaction mixture was concentrated to remove the ethyl alcohol and dissolved in dichloromethane. The extract was dried over anhydrous MgSO₄ and concentrated, giving 52 g of the (E)-(9-(2-ethoxyethyl)-6-(1-(hydroxyimino)ethyl)-9H-carbazol-3 -yl)(o-tolyl)methanone.

¹H-NMR (δ, ppm, CDCl₃): 1.06-1.08 (t, 3H), 2.3 (t, 3H), 2.7 (t, 3H), 3.4 (d, 2H), 3.8 (d, 2H), 4.5 (d, 2H), 5.2 (s, 2H), 7.3-7.4 (s, 4H), 7.5 (s, 2H), 8.0 (s, 1H), 8.1 (s, 1H), 8.5 (s, 1H), 8.6 (s, 1H)

### 3 Step: Synthesis of (E)-1-(((1-(9-(2-ethoxyethyl)-6-(2-methylbenzoyl)-9H-carbazol-3-yl)ethylidene)amino)oxy)ethanone

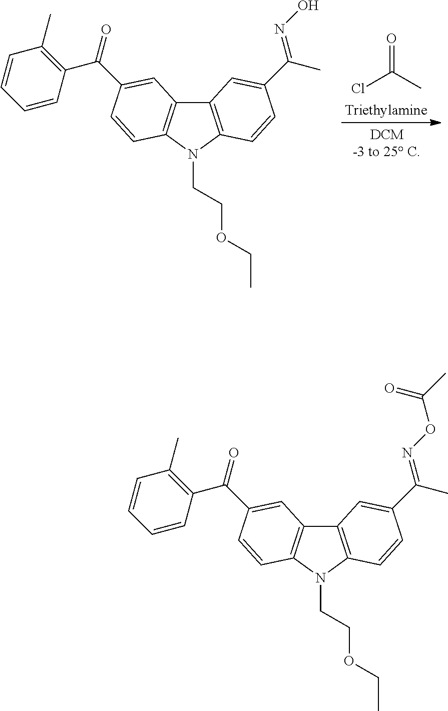

50.0 g of the (E)-(9-(2-ethoxyethyl)-6-(1-(hydroxyimino)ethyl)-9H-carbazol-3-yl)(o-tolyl)methanone was dissolved in 350 ml of dichloromethane. After the reaction mixture was cooled to 0° C., 15.2 g of triethylamine was added thereto. To the mixture was slowly added dropwise 10.5 g of acetyl chloride at 0° C. The reaction mixture was stirred at 25° C. for 2 h. The reaction mixture was washed three times with distilled water. The organic layer was dried over anhydrous MgSO₄ and concentrated. The obtained compound was isolated and purified by column chromatography, giving the (E)-1-(((1-(9-(2-ethoxyethyl)-6-(2-methylbenzoyl)-9H-carbazol-3-yl)ethylidene)amino)oxy)ethanone.

¹H-NMR (δ, ppm, CDCl₃): 1.04-1.08 (t, 3H), 2.27 (t, 3H), 2.3 (t, 3H), 2.49 (d, 2H), 3.4 (d, 2H), 3.8 (d, 2H), 4.5 (d, 2H), 7.28-7.38 (s, 4H), 7.4 (s, 2H), 7.5 (s, 1H), 7.9 (s, 1H), 8.4 (s, 1H), 8.5 (s, 1H)

### Synthesis of the Compound of Formula 5-22

### 1 Step: Synthesis of 1-(9-(2-ethoxyethyl)-6-(2-(trifluoromethyl)benzoyl)-9H-carbazol-3-yl)ethanone

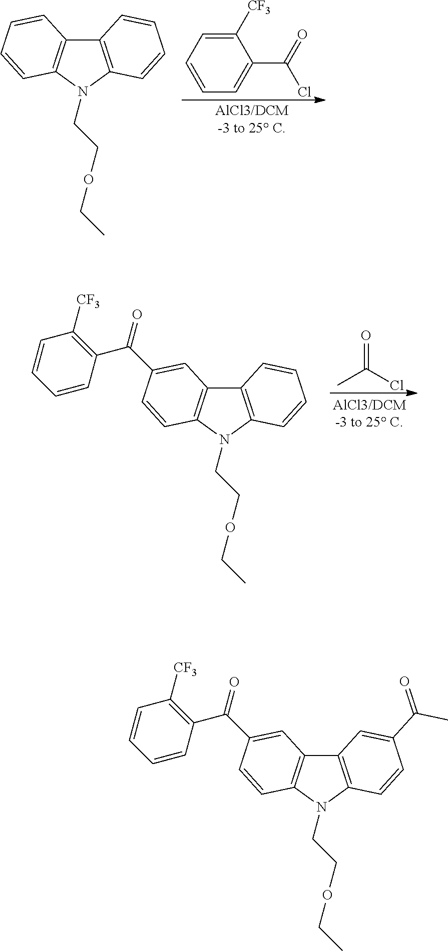

40.0 g of 9-(2-ethoxyethyl)-9H-carbazole was dissolved in 200 ml of dichloromethane. After the reaction mixture was cooled to 0° C., 16.2 g of AlCl₃ added thereto. To the reaction mixture was added dropwise 23.5 g of 2-(trifluoromethyl)benzoyl chloride at 0° C. The reaction mixture was stirred at 25° C. for 4 h. The reaction mixture was cooled to 0° C. and 16.2 g of AlCl₃ added thereto. To the mixture was slowly added 9.5 g of acetyl chloride 0° C. The reaction mixture was stirred at 25° C. for overnight. After finished the reaction, the reaction mixture was slowly added to ice-water. Following stirring for 30 min, and washed with water. The organic layer was dried over anhydrous MgSO₄ and concentrated, giving 42 g of the 1-(9-(2-ethoxyethyl)-6-(2-(trifluoromethyl)benzoyl)-9H-carbazol-3 -yl)ethanone.

¹H-NMR (δ, ppm, CDCl₃): 1.06-1.08 (t, 3H), 2.3 (t, 3H), 3.4 (d, 2H), 3.8 (d, 2H), 4.5 (d, 2H), 7.3-7.4 (s, 4H), 7.5 (s, 2H), 8.0 (s, 1H), 8.1 (s, 1H), 8.5 (s, 1H), 8.6 (s, 1H)

### 2 Step: Synthesis of (E)-(9-(2-ethoxyethyl)-6-(1-(hydroxyimino)ethyl)-9H-carbazol-3-yl)(2-(trifluoromethyl)phenyl)methanone

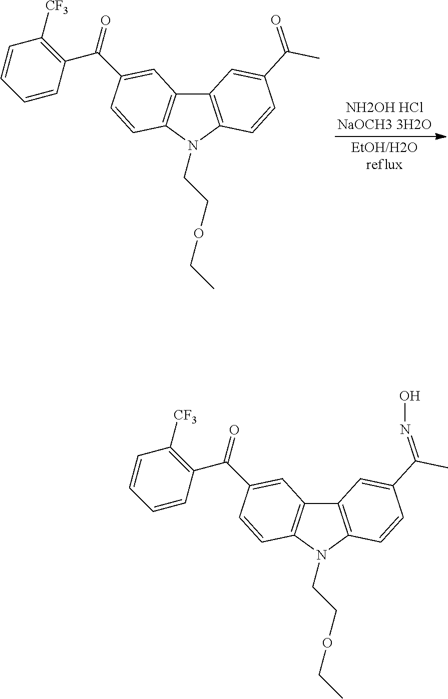 [SMILES: CCOCCn1c2ccc(C(C)=O)cc2c2cc(C(=O)c3ccccc3C(F)(F)F)ccc21 ; CCOCCn1c2ccc(C(=O)c3ccccc3C(F)(F)F)cc2c2cc(/C(C)=N/O)ccc21]

40.0 g of the 1-(9-(2-ethoxyethyl)-6-(2-(trifluoromethyl)benzoyl)-9H-carbazol-3-yl)ethanone, ethyl alcohol and water were added to dissolve the compound. To the solution was added 9.6 g of hydroxylamine chloride and 15.2 g of sodium acetate trihydrate. The reaction mixture was stirred under reflux for 5 h. After completion of the reaction, the reaction mixture was concentrated to remove the ethyl alcohol and dichloromethane was added for layer separation. The extracted layer was dried over anhydrous MgSO₄ and concentrated, giving 43 g of the (E)-(9-(2-ethoxyethyl)-6-(1-(hydroxyimino)ethyl)-9H-carbazol-3-yl)(2-(trifluoromethyl)phenyl)methanone.

¹H-NMR (δ, ppm, CDCl₃): 1.06-1.08 (t, 3H), 2.3 (t, 3H), 3.4 (d, 2H), 3.8 (d, 2H), 4.5 (d, 2H), 5.2 (s, 2H), 7.3-7.4 (s, 4H), 7.5 (s, 2H), 8.0 (s, 1H), 8.1 (s, 1H), 8.5 (s, 1H), 8.6 (s, 1H)

### 3 Step: Synthesis of (E)-1-(((1-(9-(2-ethoxyethyl)-6-(2-(trifluoromethyl)benzoyl)-9H-carbazol-3-yl)ethylidene)amino)oxy)ethanone

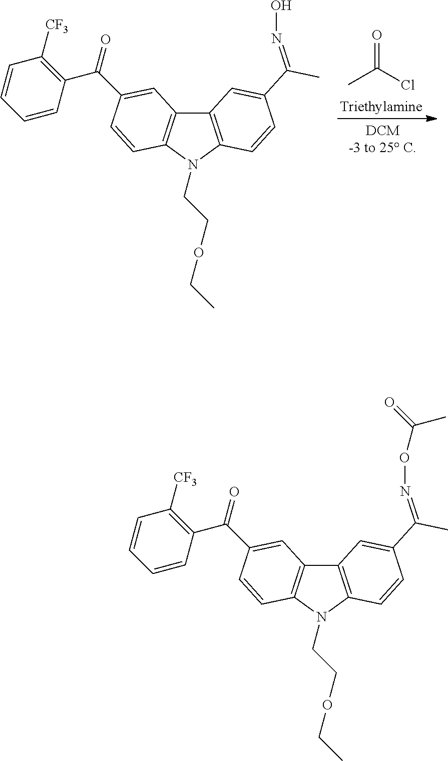

45.3 g of the (E)-(9-(2-ethoxyethyl)-6-(1-(hydroxyimino)ethyl)-9H-carbazol-3-yl)(2-(trifluoromethyl)phenyl)methanone and 450 ml of dichloromethane were placed in a reactor and 12.3 g of triethylamine was added thereto. To the mixture was added dropwise 8.9 g of acetyl chloride at 0° C. The reaction mixture was stirred for 12 h. The reaction mixture was washed three times with 450 ml of distilled water. The organic layer was concentrated. The residue was triturated ethylacetate/n-hexane to obtain a crystal. The crystal was collected by filtration to give 43 g of the (E)-1-(((1-(9-(2-ethoxyethyl)-6-(2-(trifluoromethyl)benzoyl)-9H-carbazol-3-yl)ethylidene)amino)-oxy)ethanone.

¹H-NMR (δ, ppm, CDCl₃): 1.04-1.08 (t, 3H), 2.27 (t, 3H), 2.3 (t, 3H), 2.49 (d, 2H), 3.4 (d, 2H), 3.8 (d, 2H), 4.5 (d, 2H), 7.28-7.38 (s, 4H), 7.4 (s, 2H), 7.5 (s, 1H), 7.9 (s, 1H), 8.4 (s, 1H), 8.5 (s, 1H) ¹H NMR spectroscopic data for the compounds of Formulae 5-3 to 5-26 are shown in Table 2.

TABLE 2
¹H NMR spectroscopic data

| Formula | ¹H NMR Spectrum (CDCl₃, δ) |
|---|---|
| 5-3 | 1.10 (t, 3H), 2.14 (s, 3H), 2.35 (s, 3H), 3.44 (m, 2H), 3.86 (t, 2H), 4.54 (t, 2H), |
| | 7.10-7.28 (m, 4H), 7.72 (m, 3H), 7.75 (d, 2H), 8.21-8.23 (d, 2H), 8.73 (s, 1H), 8.84 (s, 1H) |
| 5-4 | 1.09 (t, 3H), 2.53 (s, 3H), 3.41 (m, 2H), 3.85 (m, 2H), 4.54 (m, 2H), 7.10-7.28 (m, 5H), |
| | 7.53 (d, 1H), 7.58 (d, 1H), 7.60-7.69 (m, 3H), 7.75 (d, 2H), 7.98 (d, 1H), 8.11 (d, 1H), 8.23 (d, |
| | 2H), 8.50 (s, 1H), 8.72 (s, 1H) |
| 5-5 | 2.21 (s, 3H), 2.53 (s, 3H), 4.57 (d, 4H), 7.10-7.28 (m, 5H), 7.53 (d, 1H), 7.58 (d, 1H), |
| | 7.60-7.68 (m, 3H), 7.75 (d, 2H), 8.00 (d, 1H), 8.13 (d, 1H), 8.21 (d, 2H), 8.50 (s, 1H), 8.72 (s, |
| | 1H) |
| 5-6 | 2.23 (s, 3H), 2.51 (s, 3H), 3.40-3.44 (m, 2H), 3.85 (t, 2H), 5.58 (dd, 1H), 6.27 (dd, 1H), |
| | 6.03 (t, 1H), 7.27 (t, 1H), 7.53 (d, 1H), 7.58 (d, 1H), 7.75 (d, 2H), 7.98 (d, 1H), 8.11 (d, 1H), |
| | 8.50 (s, 1H), 8.72 (s, 1H) |
| 5-7 | 2.21 (s, 3H), 2.23 (s, 3H), 2.50 (s, 3H), 4.57 (d, 4H), 7.27 (t, 1H), 7.53 (d, 1H), 7.58 (d, 1H), |
| | 7.75 (d, 2H), 7.98 (d, 1H), 8.11 (d, 1H). 8.50 (s, 1H), 8.72 (s, 1H) |
| 5-9 | 2.21 (s, 3H), 2.28 (s, 3H), 2.48 (s, 3H), 4.57 (d, 4H), 7.23-7.33 (m, 4H), 7.56 (d, 1H), |
| | 7.71-7.74 (m, 2H), 7.92 (d, 1H), 8.00 (d, 1H), 8.09 (d, 1H), 8.14 (d, 1H), 8.60 (s, 1H), 8.85 (s, 1H) |
| 5-10 | 2.15 (s, 3H), 2.35 (s, 3H), 3.44 (m, 2H), 3.86 (m, 2H), 5.58 (dd, 1H), 6.27 (dd, 1H), 6.30 (t, |
| | 1H), 7.10-7.28 (m, 4H), 7.72 (m, 3H), 7.75 (d, 2H), 8.21-8.23 (d, 2H), 8.73 (s, 1H), 8.84 (s, |
| | 1H) |
| 5-11 | 0.86 (t, 3H), 1.27-1.40 (m, 6H), 1.89 (m, 2H), 2.30 (s, 3H), 2.52 (s, 3H), 7.20-7.23 (m, 1H), |
| | 7.46 (dd, 2H), 7.74 (dd, 2H), 7.98 (dd, 1H), 8.10 (dd, 1H), 8.49 (s, 1H), 8.71 (s, 1H) |
| 5-12 | 1.49 (t, 3H), 1.89 (m, 2H), 2.30 (s, 3H), 2.53 (s, 3H), 7.20-7.22 (m, 1H), 7.46 (d, 1H), |
| | 7.50 (d, 1H), 7.74 (dd, 2H), 7.99 (dd, 1H), 8.11 (dd, 1H), 8.51 (s, 1H), 8.73 (s, 1H) |
| 5-13 | 2.15 (s, 3H), 2.35 (s, 3H), 3.44 (m, 2H), 3.86 (m, 2H), 5.58 (dd, 1H), 6.27 (dd, 1H), 6.30 (t, |
| | 1H), 7.13-7.33 (m, 4H), 7.50-8.08 (m, 6H), 8.21 (s, 1H), 8.53 (s, 1H), 8.70 (s, 1H) |
| 5-14 | 2.21 (s, 3H), 2.25 (s, 3H), 2.48 (s, 3H), 4.57 (m, 4H), 7.08-7.28 (m, 4H), 7.48-8.03 (m, |
| | 10H), 8.21 (s, 1H), 8.53 (s, 1H), 8.70 (s, 1H) |
| 5-15 | 2.15 (s, 3H), 2.35 (s, 3H), 2.53 (s, 3H), 4.57 (d, 4H), 7.13-7.33 (m, 4H), 7.50-8.08 (m, 6H), |
| | 8.21 (s, 1H), 8.53 (s, 1H), 8.70 (s, 1H) |
| 5-17 | 1.49 (t, 3H), 2.14 (s, 3H), 2.35 (s, 3H), 4.43 (m, 2H), 7.13-7.33 (m, 4H), 7.50-8.08 (m, 6H), |
| | 8.21 (s, 1H), 8.53 (s, 1H), 8.70 (s, 1H) |
| 5-18 | 0.86 (t, 3H), 1.27-1.40 (m, 6H), 1.89 (m, 2H), 2.30 (s, 3H), 2.52 (s, 3H), 7.13-7.33 (m, 4H), |
| | 7.50-8.08 (m, 6H), 8.21 (s, 1H), 8.53 (s, 1H), 8.70 (s, 1H) |
| 5-21 | 1.49 (t, 3H), 2.18 (s, 3H), 2.43 (s, 3H), 4.28 (s, 2H), 4.51 (t, 2H), 7.12 (m, 5H), 7.46 (d, 1H), |
| | 7.53 (d, 1H), 7.73 (m, 2H), 7.87 (d, 1H), 8.07 (d, 1H), 8.49 (s, 1H), 8.65 (s, 1H). |
| 5-22 | 1.04-1.08 (t, 3H), 2.27 (t, 3H), 2.3 (t, 3H), 2.49 (d, 2H), 3.4 (d, 2H), 3.8 (d, 2H), 4.5 (d, 2H), |
| | 7.28-7.38 (s, 4H), 7.4 (s, 2H), 7.5 (s, 1H), 7.9 (s, 1H), 8.4 (s, 1H), 8.5 (s, 1H) |
| 5-23 | 1.04-1.08 (t, 3H), 2.27 (t, 3H), 2.3 (t, 3H), 2.49 (d, 2H), 3.4 (d, 2H), 3.8 (d, 2H), 4.5 (d, 2H), |
| | 7.14-7.27 (m, 4H), 7.34 (m, 2H), 7.40 (m, 2H), 8.65 (s, 1H), 8.84 (s, 1H) |
| 5-24 | 1.04-1.08 (t, 3H), 2.27 (t, 3H), 2.3 (t, 3H), 2.49 (d, 2H), 3.4 (d, 2H), 3.8 (d, 2H), 4.5 (d, 2H), |
| | 5.73 (s, 1H), 7.14-7.27 (m, 4H), 7.34 (m, 2H), 7.40 (m, 2H), 7.61-7.66 (m, 4H), 8.65 (s, |
| | 1H), 8.84 (s, 1H) |
| 5-25 | 1.10 (t, 3H), 2.28 (s, 3H), 3.42 (m, 2H), 3.83 (t, 2H), 4.17 (m, 2H), 4.52 (t, 2H), 5.73 (m, |
| | 1H), 7.14-7.27 (m, 4H), 7.34 (m, 2H), 7.40 (m, 2H), 7.61-7.66 (m, 4H), 8.65 (s, 1H), |
| | 8.84 (s, 1H) |
| 5-26 | 1.10 (t, 3H), 1.89 (t, 2H), 2.27 (s, 3H), 2.71 (t, 2H), 3.06 (t, 2H), 3.42 (m, 2H), 3.83 (t, 2H), |
| | 4.52 (t, 2H), 7.33-7.39 (m, 5H), 7.28 (dd, 1H) 7.47 (d, 1H), 7.50 (d, 1H), 7.99 (d, 1H), |
| | 8.09 (d, 1H), 8.14 (d, 1H), 8.59 (s, 1H), 8.83 (s, 1H) |

### EXAMPLE 3

Preparation of Transparent Resist Composition

A mixture of 17 g of an acrylic copolymer, 13.6 g of dipentaerythritol hexaacrylate, 1.5 g of the compound of Formula 4-1 as a photoinitiator, and 67 g of propylene glycol monoethyl ether was thoroughly stirred to prepare a transparent resist composition.

### EXAMPLE 4

Preparation of Black Resist Composition

A mixture of 10 g of an acrylic copolymer, 13.6 g of dipentaerythritol hexaacrylate, 2.0 g of the compound of Formula 4-1 as a photoinitiator, 48 g of Pigment Black 7, and 25 g of propylene glycol monoethyl ether was thoroughly stirred to prepare a black resist composition.

### EXAMPLE 5

Preparation of Red Resist Composition

A mixture of 10 g of an acrylic copolymer, 13.6 g of dipentaerythritol hexaacrylate, 1.5 g of the compound of Formula 4-1 as a photoinitiator, 25 g of Pigment Red 192, and 49 g of propylene glycol monoethyl ether was thoroughly stirred to prepare a red resist composition.

### EXAMPLE 6

Preparation of Transparent Resist Composition

A mixture of 17 g of an acrylic copolymer, 13.6 g of dipentaerythritol hexaacrylate, 1.5 g of the compound of Formula 5-1 as a photoinitiator, and 67 g of propylene glycol monoethyl ether was thoroughly stirred to prepare a transparent resist composition.

### EXAMPLE 7

Preparation of Black Resist Composition

A mixture of 10 g of an acrylic copolymer, 13.6 g of dipentaerythritol hexaacrylate, 2.0 g of the compound of Formula 5-1 as a photoinitiator, 48 g of Pigment Black 7, and 25 g of propylene glycol monoethyl ether was thoroughly stirred to prepare a black resist composition.

### EXAMPLE 8

Preparation of Red Resist Composition

A mixture of 10 g of an acrylic copolymer, 13.6 g of dipentaerythritol hexaacrylate, 1.5 g of the compound of Formula 5-1 as a photoinitiator, 25 g of Pigment Red 192, and 49 g of propylene glycol monoethyl ether was thoroughly stirred to prepare a red resist composition.

### COMPARATIVE EXAMPLE 1

Preparation of Transparent Resist Composition

A mixture of 17 g of an acrylic copolymer, 13.6 g of dipentaerythritol hexaacrylate, 1.5 g of the compound of Formula 6 as a photoinitiator, and 67 g of propylene glycol monoethyl ether was thoroughly stirred to prepare a transparent resist composition.

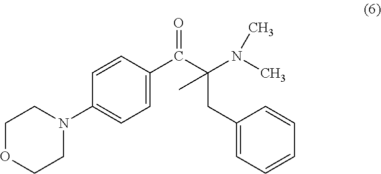 [SMILES: CN(C)C(C)(Cc1ccccc1)C(=O)c1ccc(N2CCOCC2)cc1]

### COMPARATIVE EXAMPLE 2

Preparation of Black Resist Composition

A mixture of 10 g of an acrylic copolymer, 13.6 g of dipentaerythritol hexaacrylate, 2.0 g of the compound of Formula 6 as a photoinitiator, 48 g of Pigment Black 7, and 25 g of propylene glycol monoethyl ether was thoroughly stirred to prepare a black resist composition.

### COMPARATIVE EXAMPLE 3

Preparation of Red Resist Composition

A mixture of 10 g of an acrylic copolymer, 13.6 g of dipentaerythritol hexaacrylate, 1.5 g of the compound of Formula 6 as a photoinitiator, 25 g of Pigment Red 192, and 49 g of propylene glycol monoethyl ether was thoroughly stirred to prepare a red resist composition.

### COMPARATIVE EXAMPLE 4

Preparation of Transparent Resist Composition

A mixture of 17 g of an acrylic copolymer, 13.6 g of dipentaerythritol hexaacrylate, 1.5 g of the compound of Formula 7 as a photoinitiator, and 67 g of propylene glycol monoethyl ether was thoroughly stirred to prepare a transparent resist composition.

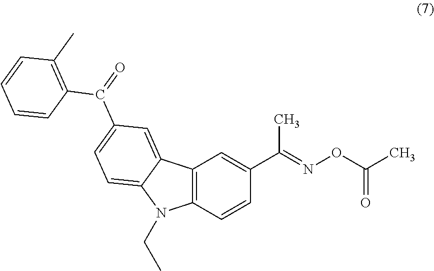 [SMILES: CCn1c2ccc(C(=O)c3ccccc3C)cc2c2cc(/C(C)=N/OC(C)=O)ccc21]

### COMPARATIVE EXAMPLE 5

Preparation of Black Resist Composition

A mixture of 10 g of an acrylic copolymer, 13.6 g of dipentaerythritol hexaacrylate, 2.0 g of the compound of Formula 7 as a photoinitiator, 48 g of Pigment Black 7, and 25 g of propylene glycol monoethyl ether was thoroughly stirred to prepare a black resist composition.

### COMPARATIVE EXAMPLE 6

Preparation of Red Resist Composition

A mixture of 10 g of an acrylic copolymer, 13.6 g of dipentaerythritol hexaacrylate, 1.5 g of the compound of Formula 7 as a photoinitiator, 25 g of Pigment Red 192, and 49 g of propylene glycol monoethyl ether was thoroughly stirred to prepare a red resist composition.

The characteristics of the photosensitive compositions were evaluated in the following test examples.

### TEST EXAMPLES

Each of the photosensitive compositions was applied to a spin coater at 800-900 rpm for 15 sec and dried on a hot plate at 90° C. for 100 sec. The photosensitive composition was exposed to an ultra-high pressure mercury lamp as a light source through a patterned mask, spin developed in a 0.04% potassium hydroxide solution at 25° C. for 60 sec, washed with water, dried, and baked at 230° C. for 40 min to form a pattern. The pattern was evaluated for the following properties. The photoinitiators used in the photosensitive compositions and the results of evaluations are shown in Table 3.

(1) Adhesiveness

The adhesiveness of each of the photosensitive compositions was evaluated in accordance with JIS D 0202 standard test method. First, the photosensitive composition was coated, exposed, developed, and heated at 200° C. for 30 min to form a film. After 100 cross-cuts in the shape of grids were scribed on the film, a peeling test was conducted using a cellophane tape. The peeling state of the grid-shaped cross-cuts was observed. The adhesiveness was judged to be “∘” when 95 or more grids were not peeled, “Δ” when 60 or more grids were not peeled, and “×” when 40 or more grids were peeled.

(2) Alkali Resistance

Each of the photosensitive compositions was developed and baked at 230° C. for 30 min to form a film. The film was sequentially dipped in a 5% aqueous NaOH solution for 24 h, in a 4% aqueous KOH solution at 50° C. for 10 min, and in a 1% aqueous NaOH solution at 80° C. for 5 min. The state of the film after dipping was observed. The alkali resistance of the film was judged to be “∘” when neither any change in appearance nor peeling was observed, “Δ” when resist curling was observed, and “×” when resist peeling was observed.

(3) Sensitivity Evaluation

Each of the photosensitive resin compositions was applied to a glass substrate (Eagle2000, Samsung Corning) using a spin coater and dried on a hot plate at 90° C. for 1 min. The film thicknesses of the black resist and the transparent negative resist were 1 microns and 5 microns, respectively, as measured using a stylus profilometer (α-step 500, KLA-Tencor). Next, each sample was exposed to a high pressure mercury lamp through a mask. Thereafter, the exposed sample was developed with a 0.04% aqueous potassium hydroxide solution by spraying to obtain a resist pattern. An optimum exposure dose (mJ/cm²) at which the resist pattern reached dimensions corresponding to those of the 40-micron mask pattern was defined as the sensitivity of the sample. That is, a resist composition requiring a lower exposure dose can be patterned with less light energy, indicating higher sensitivity.

(4) Whitening Phenomenon

Each of the photosensitive resin compositions including the photoinitiators was applied to a glass substrate using a spin coater. Depending on the solubility of the photoinitiator, a crystal was formed during spin coating. The surface state of the film was judged to be “×” when a crystal was formed to make the coated surface very defective, “Δ” when a crystal was formed to make the surface hazy, and “∘” when the photoinitiator was well dissolved in the resist composition, leaving a clean surface on which no crystal was formed.

The results are shown in Table 3.

TABLE 3

| | | | | | Thin film |
|---|---|---|---|---|---|
| | | | Alkali | Sensitivity | characteristics |
| Example No. | Photoinitiator | Adhesiveness | resistance | (mJ/cm²) | (Whitening) |
| Example 3 | Formula 4-1 | ∘ | ∘ | 30 | ∘ |
| Example 4 | Formula 4-1 | ∘ | ∘ | 55 | ∘ |
| Example 5 | Formula 4-1 | ∘ | ∘ | 40 | ∘ |
| Example 6 | Formula 5-1 | ∘ | ∘ | 40 | ∘ |
| Example 7 | Formula 5-1 | ∘ | ∘ | 65 | ∘ |
| Example 8 | Formula 5-1 | ∘ | ∘ | 55 | ∘ |
| Comparative | Formula 6 | Δ | Δ | 100 | ∘ |
| Example 1 | | | | | |
| Comparative | Formula 6 | x | x | 150 | x |
| Example 2 | | | | | |
| Comparative | Formula 6 | Δ | Δ | 120 | Δ |
| Example 3 | | | | | |
| Comparative | Formula 7 | Δ | Δ | 80 | ∘ |
| Example 4 | | | | | |
| Comparative | Formula 7 | x | x | 120 | x |
| Example 5 | | | | | |
| Comparative | Formula 7 | Δ | Δ | 100 | Δ |
| Example 6 | | | | | |

As can be seen from the results in Table 3, the inventive photosensitive compositions, each of which included the oxime ester compound or the α-ketoxime ester compound, were excellent in terms of adhesiveness and alkali resistance and did not undergo whitening upon film formation. Particularly, the photosensitive resin compositions of Examples 3-5, each of which used the α-ketoxime ester initiator, could be patterned when irradiated with light at exposure doses as low as 30-55 mJ/cm², indicating very high sensitivities. In addition, the highly sensitive photosensitive compositions, each of which used the oxime ester compound or the α-ketoxime ester compound, had high degrees of cure, ensuring good adhesion to the substrates and good resistance to the basic aqueous solutions. Furthermore, high compatibility between the binder and the polyfunctional monomer with an ethylenically unsaturated bond used in the inventive photosensitive compositions and high solubility of the inventive photosensitive compositions in the organic solvents enabled the formation of thin films with very uniform surfaces.

1. A photoinitiator represented by Formula 1: 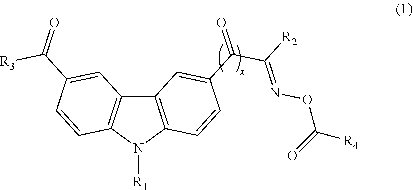
wherein R₁ is a C₁-C₁₂ linear, branched or cyclic alkyl group optionally containing oxygen, sulfur, nitrogen or an ester bond in the chain,
R₂ is a C₁-C₆ alkyl group; a C₆-C₂₀ aryl group optionally substituted with oxygen, sulfur, nitrogen, a C₁-C₃ alkyl group, a nitro group or a halogen atom;
a 2-methylbenzyl group; 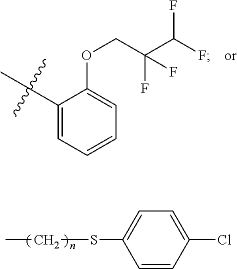
(n=1-4),
R₃ is a C₁-C₁₀ linear, branched or cyclic alkyl group, a C₆-C₂₀ aryl group, or a C₄-C₂₀ heteroaryl group,
R₄ is a C₁-C₁₀ linear, branched or cyclic alkyl group or a phenyl group, and
x is 0 or 1.

2. The photoinitiator according to claim 1, wherein R₁ is a C₁-C₁₂ alkoxyalkyl or acyloxyalkyl group.

3. The photoinitiator according to claim 2, wherein x is 1.

4. The photoinitiator according to claim 2, wherein R₃ is a thienyl, naphthyl, tolyl, or C₆-C20 aryl group in which the aryl group is optionally substituted with a fluoro group, a fluorinated alkyl group, or a fluorinated alkoxy group.

5. The photoinitiator according to claim 1, wherein the photoinitiator of Formula 1 is a compound represented by Formula 2 or 3:

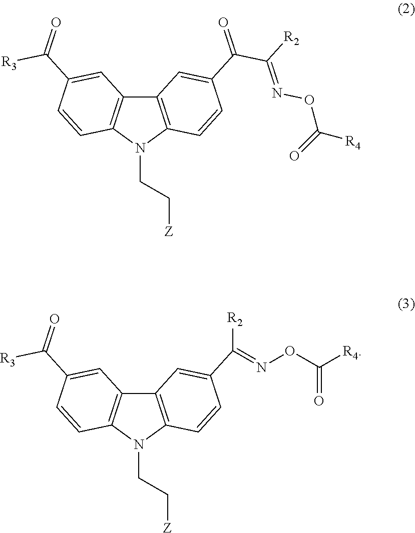

6. The photoinitiator according to claim 1, wherein Z is —H, —R₅, —OR₅, —OC(O)R₅, —C(O)OR₅ or —OC(O)OR₅, R₃ is a thienyl, naphthyl, tolyl, 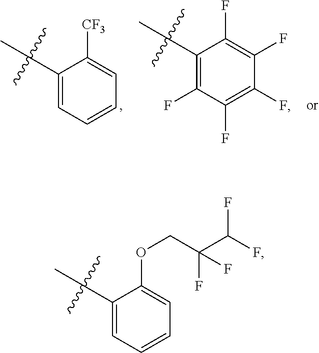 [SMILES: *c1ccccc1C(F)(F)F ; "," ; *c1c(F)c(F)c(F)c(F)c1F ; "or" ; *c1ccccc1OCC(F)(F)C(F)F]
and R₅ is C₁-C₆ linear, branched or cyclic alkyl,

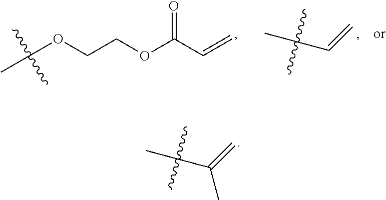

7. The photoinitiator according to claim 6, wherein R₂ is methyl, tolyl, 2-methylbenzyl, 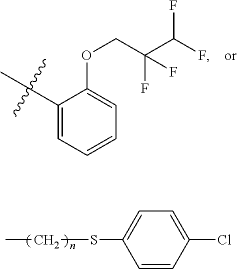
(n=1-4) and R₄ is methyl or phenyl, Z is —OR₅ or —OC(O)R₅.

8. A photosensitive resin composition comprising the photoinitiator according to claim 1.
